(19) United States
(12) Patent Application Publication
(10) Pub. No.: US 2006/0154237 A1
(43) Pub. Date: Jul. 13, 2006

(54) Soluble rna polymerase protein and methods for the use thereof

(75) Inventors: Eugene Makeyev, Cambridge, MS (US); Dennis Bamford, Helsinki (FI)

Correspondence Address: FOLEY AND LARDNER LLP;SUITE 500, 3000 K STREET NW, WASHINGTON, DC, 20007, US

(73) Assignee: RNA-Line Oy

(21) Appl. No.: 10/532,053

(22) PCT Filed: Oct. 17, 2003
(86) PCT No.: PCT/FI03/00776

§ 371 Date: Mar. 7, 2006

Related U.S. Application Data
(60) Provisional application No. 60/419,562, filed on Oct. 21, 2002.

(51) Int. Cl. C12Q 1/70; C12Q 1/68; C12P 19/34; C12N 9/22

(52) U.S. Cl. 435/5; 435/91.2; 435/6; 435/199

(57) ABSTRACT

A polymerase protein originating from a eukaryotic cell and involved in the RNA silencing pathway is for the first time provided in a purified soluble form that possesses a detectable RNA polymerization activity. This polymerase is useful in methods and kits for in vitro RNA synthesis. A polymerase of the invention copies ssRNA templates to produce two types of reaction products: short and long RNA copies. It can also copy ssDNA templates. The polymerization does not require a primer for the initiation of RNA synthesis, although RNA synthesis can be also initiated in the presence of a primer. In addition to standard nucleotides polymerase of this invention also accepts a number of modified nucleotides. The polymerase is useful in many downstream applications such as production of labeled RNA probes or generation of trigger RNA molecules to induce RNA interference effects in living cells or suitable in vitro systems.

### FIELD OF THE INVENTION

This invention relates, in general, to enzymatic synthesis of RNA using nucleic acid templates. More specifically, the invention deals with RNA synthesis catalyzed by a cellular RNA polymerase that is involved in the posttranscriptional gene silencing process. The invention discloses a method for producing a nucleic acid product by using said polymerase, a soluble and active form of said polymerase and nucleic acid sequences encoding said soluble active polymerase. Methods and kits for RNA synthesis by contacting said polymerase with nucleic acid templates are also disclosed. The invention also relates to downstream applications of the RNA-polymerization products.

### BACKGROUND OF THE INVENTION

Term post-transcriptional gene silencing (PTGS), or RNA silencing, refers to a group of sequence-specific mRNA degradation mechanisms in eukaryotic cells (Baulcombe, 2002). First discovered in plants under the names of co-suppression, PIGS and virus induced gene silencing, this phenomenon has been reported for the filamentous fungus Neurospora crassa as quelling, and for a number of animals and protozoa as RNA interference (RNAi) (Cogoni and Macino, 1999; Fire, 1999; Fjose et al., 2001). RNA silencing is induced and mediated by double-stranded (ds) RNA triggers sufficiently homologous to the target template. It is used as a system of cell defense against viral RNAs, transposons and, under experimental conditions, transgenes and synthetic dsRNAs (Plasterk, 2002; Waterhouse et al., 2001).

RNA silencing is of growing practical importance. Transgene-induced PTGS has been used in agriculture e.g. to change petal color in petunia or delay maceration in tomatoes (Baulcombe, 2002). Sequence-specific dsRNAs are being employed for research purposes to silence expression of cognate genes in flies (e.g. Drosophila melanogaster) and worms (e.g. Caenorhabditis elegans). In the latter case, high-throughput efforts have been reported, where functions of thousands of genes have been screened using the RNAi (Barstead, 2001; Fraser et al., 2000; Gonczy et al., 2000; Maeda et al., 2001). Finally, recent work demonstrates that RNA silencing can be used to interfere with poliovirus and human immunodeficiency virus (HIV) replication in mammalian cells, which opens up new avenues in medicine (Gitlin et al., 2002; Jacque et al., 2002; Novina et al., 2002).

The practical utility of RNA silencing would not be possible without recent advances in clarifying the molecular details of this phenomenon. One important step along this way was the observation that dsRNA molecules injected or even fed to the nematode Caenorhabditis elegans brought about the degradation of homologous host-encoded mRNAs (RNAi; (Fire et al., 1998; Montgomery et al., 1998; Timmons and Fire, 1998). Then, it was discovered that the PTGS in plants is invariably linked with the accumulation of sequence-specific ˜25 nt long RNAs of both sense and antisense polarity (Hamilton and Baulcombe, 1999). The possible role of the short RNAs became apparent when it was shown that double-stranded RNAi triggers are first cleaved by the dsRNA-specific nuclease, Dicer, into 21-23 nt long dsRNAs with several-nucleotide long 3′-protruding ends (Bernstein et al., 2001; Zamore et al., 2000). These, sometimes called small interfering RNAs (siRNAs), are then used to guide via Watson-Crick base-pairing another ribonuclease complex, RISC, to the complementary mRNAs ultimately causing its degradation (Elbashir et al., 2001; Hammond et al., 2000). In addition to plants and animals, 23-25 nt RNAs have been recently described for Dictyostelium and N. crassa, thus suggesting the conservation of PIGS mechanisms across the eukaryotes (Catalanotto et al., 2002; Martens et al., 2002).

Although dsRNA mediators are now accepted as a paradigm of RNA silencing, the way they appear in the PTGS-committed cell remains largely unknown. They can arise as replication intermediates of RNA viruses or intramolecular hairpin-like transcripts produced from inverted repeat sequences. However, this does not explain the wide range of cases when RNA silencing is triggered by overexpression of ectopically inserted transgenes (co-suppression; (Cogoni and Macino, 1999). It has been proposed that co-suppression and related RNA silencing phenomena can be induced by aberrant ssRNAs (abRNAs) that are converted into dsRNA triggers by cellular RNA polymerases (Wassenegger and Pelissier, 1998). However, the nature of the abRNAs, as well as the details of their transformation into the double helix, are not understood.

Genetic screens carried out in N. crassa have identified three loci essential for PTGS: qde-1, qde-2 and qde-3 (from quelling defective; Cogoni and Macino, 1997). In plants and animals the genetic repertoires of PTGS are more complex, yet often containing homologs of the qde genes (Hutvagner and Zamore, 2002; Waterhouse et al., 2001). Of these, qde-3 is a member of the RecQ/WRN helicase family that also includes mut-7 of the C. elegans RNAi pathway (Cogoni and Macino, 1999; Ketting et al., 1999). The gene product of qde-2 belongs to the eIF2C/AGO1/RDE-1/Piwi/Zwille group of RNA-binding proteins (Cogoni and Macino, 2000; Fagard et al., 2000; Tabara et al., 1999).

The gene qde-1 contains homology to the tomato gene encoding virus/viroid induced RNA-dependent RNA polymerase (RdRP). Tomato RdRP is so far the only cellular RdRP with biochemically shown RNA-synthesizing activity (Schiebel et al., 1993; Schiebel et al., 1993; Schiebel et al., 1998). However, it is presently unknown if this protein is associated with the PTGS process. Furthermore, despite considerable efforts it has been impossible thus far to produce enzymatically active tomato RdRP from a recombinant source (Schiebel et al., 1998). The currently used procedure for providing this protein in the form suitable for enzymatic assays is expensive and time-consuming. Hundreds of grams to kilograms of viroid-infected tomato leaves are needed as starting material and the purification protocol includes a number of chromatography steps in addition to the lysate clarification and ammonium sulfate precipitation steps (Schiebel et al., 1993; and see U.S. Pat. No. 6,218,142).

Interestingly, sequence homologs of tomato RdRP are also found in many other organisms and very often there is more than one RdRP-like gene per genome. Four such genes are known in C. elegans, three in Dictyostelium and seven in Arabidopsis (Martens et al., 2002; Mourrain et al., 2000; Smardon et al., 2000). Thus far, only the qde-1 of N. crassa, ego-1 and rrf-1 of C elegans, RrpA of Dictyostelium and SGS2/SDE1 of Arabidopsis have been genetically linked with RNA silencing, mutations in other RdRP genes having little or no effect on PTGS phenotypes (Cogoni and Macino, 1999; Dalmay et al., 2000; Martens et al., 2002; Mourrain et al., 2000; Sijen et al., 2001; Smardon et al., 2000). Notably, the second RdRP-like gene of N. crassa, sad-1, is shown to function in yet to be characterized meiotic silencing pathway (Shiu et al., 2001). Apparently, different RdRP-like proteins from a single organism can play distinct functions, one of these functions being connected to PTGS.

Several hypothetical models have been proposed for the role of RdRP in RNA silencing (FIG. 1A). However, the verification of these models has not been possible due to the unavailability of the corresponding RdRP-like proteins in purified active form. It has not been known if these components of PTGS can indeed catalyze RNA synthesis, since the tomato RdRP prototype has never been linked with RNA silencing. Given the importance of PTGS and related phenomena for life sciences, medicine and agriculture, there is an urgent need in the art for a methodology that would allow one to obtain sufficient amounts of biologically active RdRP-like proteins from RNA silencing pathway and determine their enzymatic properties in vitro. It would be highly advantageous to isolate said active RdRP-like proteins from a recombinant source, because this would enable both high protein yields and the possibility of knowledge-based genetic engineering.

Importantly, currently preferred methods for inducing RNA silencing (RNAi) in eukaryotic organisms are relatively laborious (for the RNAi protocols see e.g. Barstead, 2001; Fraser et al., 2000; Gonczy et al., 2000; Maeda et al., 2001). RNAi in C. elegans and a number of other organisms is presently done by first providing DNA fragments encoding genes of interest, transcribing said DNA fragments in two transcription reactions (using e.g. opposing T7 and T3 RNA polymerase promoters), annealing the two complementary RNA transcripts to form dsRNA trigger, and finally delivering the dsRNA into the organism or cell to induce RNAi. It would be highly advantageous, especially for high-throughput RNAi projects, to be able to produce dsRNA triggers from their encoding DNA templates in a simpler way.

The present invention discloses the efficient production of a recombinant RdRP protein involved in RNA silencing and its genetically altered derivatives. We also report for the first time a procedure for providing said recombinant RdRP and its derivatives in purified, soluble form. Further disclosed are methods and kits for using RNA-dependent RNA polymerization activity of these proteins with different templates using either de novo or primer-dependent initiation modes. Unlike the earlier described RdRP preparation from tomato, the enzyme of this invention synthesizes two distinct types of RNA products: (1) extensive (full-length or nearly full-length) copies and (2) short 7-40 nt, mostly within 9-21 nt long RNAs base-paired with the template along the entire template length. The latter type of products has been never described for an RdRP. The invention explains how this unique reaction mode can be used for the benefit of several downstream applications including RNAi and microarray technology.

### SUMMARY OF THE INVENTION

This invention discloses RNA polymerization processes using a newly isolated recombinant polymerase from the PTGS pathway. Said polymerase and its derivatives are provided in soluble, active form suitable for in vitro assays. Methods and kits for RNA synthesis by contacting said polymerase or its derivatives with different templates are disclosed. The invention also discloses downstream applications of the RNA-polymerization products, such as inducing RNA silencing in living cells and cell-free extracts or the synthesis of labeled RNA probes suitable for research and diagnostic purposes. Other features, aspects and advantages of the present invention will become apparent from the following description and appended claims.

### BRIEF DESCRIPTION OF THE FIGURES

The foregoing text, as well as the following detailed description of the present invention, will be better understood when read in conjunction with the appended figures, in which

FIG. 1 depicts:

(A) Three hypothetical models for the role of RdRP in RNA silencing that have been suggested in prior art, but not tested experimentally. In the first model, RdRP is thought to replicate dsRNA templates, thus intensifying the silencing signal (Waterhouse et al., 1998). A variant of this model suggests that dsRNA templates can be used by RdRP to generate multiple copies of shorter RNA triggers (“diffusible silencing factors”; (Chicas and Macino, 2001).

According to the second model, RdRP also increases the concentration of a dsRNA trigger, but the amplification is achieved through extending 3′ termini of RNA primers complementary to mRNA, rather than dealing with dsRNA templates (Nishikura, 2001). Small antisense RNAs produced by dicing the initial dsRNA trigger can be used as primers that would anneal to many mRNA templates and, after the RdRP extension step, give rise to a plurality of the secondary dsRNA triggers. Recent work on C. elegans has demonstrated that these secondary triggers do exist and that their synthesis requires the presence of a functional rrf-1 gene (Sijen et al., 2001). But the involvement of primers has not been shown in this case directly. So far the only unequivocal evidence for the primer-dependent synthesis of secondary dsRNAs comes from a study carried out in D. melanogaster embryo extracts (Lipardi et al., 2001). Interestingly, the Drosophila genome contains no apparent homologs of the cellular RdRP family, which implies that some other polymerase is responsible for the observed effects.

Finally, rather than being an ancillary amplification device that increases dsRNA concentration, RdRP may be needed for the synthesis of the initial dsRNA triggers from ssRNAs (the third model; Cogoni and Macino, 2000; Wassenegger and Pelissier, 1998). This model would explain how aberrant transcripts and some viral ssRNAs might be converted into the double-stranded silencing triggers.

(B) Similarity profile for the five RdRP-like proteins with genetically documented role in PTGS and their biochemical counterpart isolated from tomato leaves (T-RdRP). The graph is generated in the AlignX program of the Vector NTI Suite using a 40 aa sliding window. The protein sequences can be accessed at http://www.ncbi.nlm.nih.gov/ under the following numbers: QDE-1, CAB42634; RrpA, CAC41974; EGO-1, AAF80367; RRF-1, AAF80368; SGS2/SDE1, AAF74208; T-RdRP, CAA71421. The 1-442 aa fragment of RrpA homologous to the Dicer helicase domain was excluded from the alignment. Regions with <20% similarity are colored gray; ≧20% similarity, black. HS refers to the most conserved span within the family of cellular RdRPs. The inset shows a phylogenetic tree for the six deduced protein sequences built using the neighbor-joining method (Saitou and Nei, 1987).

(C) Protein sequence alignment for the HS span. Black shades, invariant residues; gray shades, conserved residues. The aspartate with proposed catalytic role is marked with the asterisk.

FIG. 2 depicts purification of QDE-1 and its genetic derivatives and initial RNA-polymerization assays.

(A) SDS-PAGE analysis of purified QDE-1 (lane 1), ΔN (lane 2) and ΔN^{DA} (lane 3). M, marker lane. Molecular masses of the protein standards are indicated on the right. Sequence deduced molecular masses of His-tagged QDE-1 and ΔN (ΔN^{DA}) are ˜163 and ˜122 kDa, respectively. (B) The purified proteins were assayed in 10 μl mixtures containing 90 μg/ml of firefly luciferase (luc) mRNA (T7 transcript of pT7 luc cut with HindIII; (Kolb et al., 2000) in the presence of the four unlabeled NTPs and [α-³²P]UTP and the reaction products were analyzed by native agarose. gel-electrophoresis. Lanes, 1, M-200 buffer control; 2, reaction containing 10 μg/ml QDE-1; 3, 10 μg/ml ΔN; 4, 10 μg/ml ΔN^{DA}; 5, 100 μg/ml ΔN^{DA}. M^{ds} is dsDNA marker. Upper panel, ethidium bromide (EtBr) stainied gel; lower panel, autoradiogram. Positions of ss and the full-length ds forms of the template RNA are shown on the left. Marker lengths are indicated on the right. (C) A₂₈₀ elution profile of ΔN (30 μg) separated on a Superdex 200 column (Pharmacia; 20 mM Tris-HCl pH 8.9, 100 mM NaCl; 1 ml/min; 1 ml fractions). Arrows show the positions of gel-filtration markers (Sigma): BD, blue dextran; βAM, β-amylase; AD, alcohol dehydrogenase; BSA, bovine serum albumin; CA, carbonic anhydrase; CC, cytochrome C. Peak at 20 min corresponds to nonionic detergents from the RdRP storage buffer. Inset, SDS-PAGE analysis of the eluate fractions. (D) RdRP activity measured in the Superdex 200 fractions using luc RNA template. Note that the activity peak corresponds to the position of ΔN protein (fractions 12-14).

FIG. 3 shows that QDE-1 catalyzes RNA-dependent RNA polymerization.

(A) QDE-1 (4 μg/ml) was incubated in mixtures containing either all four NTPs (1 mM of ATP and GTP and 0.2 mM of CTP and UTP, lanes 2-3) or only 0.2 mM of UTP (lane 4) in the presence of a constant amount of [α-³²P]UTP. 40 μg/ml of bacteriophage φ6 RdRP (lane 1; φ6Pol) was assayed as a control in the presence of the four NTPs. In all cases, except lane 2, mixtures contained 90 μg/ml luc RNA. Reaction products were separated in 1% agarose gel under native conditions. Positions of the ss and full-length ds forms of the luc RNA are shown on the left. (B) RdRP reactions containing 10 μg/ml of QDE-1, 0.2 mM each of the four unlabeled NTPs and [α-³²P]UTP were programmed with either 90 μg/ml luc RNA (track 2), 150 μg/ml poly(A) homopolymer (Sigma; track 3), or no RNA (track 1). Reaction products were purified from unincorporated nucleotides by gel-filtration and subjected to RNase T2 digestion and TLC separation as described (Schiebel et al., 1993). Black arrows indicate position of the four nucleoside-3′-monophosphates. The open arrow shows the TLC developing direction. (C) EtBr-stained agarose gel (lanes 1-4) and corresponding autoradiogram (1*-4*) for RdRP reactions carried out with 100 μg/ml 5′Δmₛ⁺ RNA in the presence of [γ-³²P]GTP (lanes 1, 2, 4 and 1*, 2*, 4*) or [γ-³²P]ATP (lane 3 and 3*). Lanes: 1 (1*) and 3 (3*), 20 μg/ml ΔN polymerase; 2 (2*), 20 μg/ml φ6Pol; 4, M-200 buffer. Other designations as in FIG. 2B. (D) Reactions done as in (C) were purified from unincorporated nucleotides, treated with RNase T1 and separated by TLC. Tracks: 1, no RNA; 2, luc RNA; 3, a control RNA oligonucleotide with the 5′ terminal G labeled with T4 PNK and [γ-³²P]ATP. Black arrows, positions of the guanosine phosphates; open arrow, TLC developing direction. (E) The effect of divalent metal ions on the QDE-1 (4 μg/ml) catalyzed reaction. Lane 1, 5 mM MgCl₂; other reactions additionally contained 1, 2 or 4 mM of MgCl₂ (lanes 2-4), MnCl₂ (lanes 5-7), or CaCl₂ (lanes 8-10).

FIG. 4 shows that QDE-1 generates two types of reaction products.

(A-B) RdRP reactions were programmed with 90 μg/ml luciferase mRNA (lanes 1-3), 80 μg/ml of GFP mRNA (lanes 4-6; T7 transcript of plasmid TU58; (Chalfie et al., 1994) cut with EcoRI) or 100 μg/ml of TMV genomic RNA (lanes 7-10). Reactions contained [α-³²P]UTP and either 10 μg/ml QDE-1 (lanes 2, 5, 8) or 20 μg/ml φ6Pol. Lanes 1, 4, 7 are “buffer only” controls. (A) native 1.2% agarose gel. Short RNA products (sRNAs) are indicated with the open arrowhead. dsDNA marker position are shown on the right. (B) formaldehyde-containing 1.5% agarose gel. Positions of single-stranded templates used for the RdRP reaction are shown on the left. M^{ss}, two ³²P-labeled ssRNA markers (20 and 1797 nt); (C) Schematic and (D) actual results of the RNase protection assay. RdRP reactions were carried out with 70 μg/ml of ³²P-labeled luc mRNA and no labeled NTPs. Reactions contained 40 μg/ml φ6Pol (lanes 1, 4), 40 μg/ml QDE-1 (lanes 2, 5), or M-200 buffer (lanes 3, 6). On 1 h incubation at 30° C., polymerization was stopped by EDTA and aliquots were incubated with RNase I (lanes 4-6) or RNase I reaction buffer (lanes 1-3), as specified under Experimental Procedures. The black arrowhead indicates the position of short dsRNA fragments in lane 5. The faint slow-migrating band in lane 3 apparently represents a conformer of luc mRNA.

FIG. 5 depicts size distribution of sRNAs.

Reaction were carried out as in FIG. 4A-B but in the presence of [γ-³²P]GTP and the additives indicated on the top of the panel. Aliquots were withdrawn at 15 min (lanes 1-6) and 60 min (lanes 7-15) time points and analyzed by 15% urea-containing PAGE. M, ³²P-labeled ssRNA markers produced by T7 transcription of a mixture of DNA templates. Marker positions are indicated on the right (in nt). The arrow indicates the position of 21-mer RNAs. Only the lower half of the gel is presented.

FIG. 6 shows that QDE-1 accepts a variety of ssRNA templates.

(A) RdRP reactions were carried out with (even lanes) or without (odd lanes) 10 μg/ml QDE-1, in the presence of [α-³²P]UTP and various ssRNAs, and analyzed by native agarose gel-electrophresis. Upper panel, EtBr staining; lower panel, autoradiogram. The sizes of dsDNA markers (M^{ds}) are shown on the right. Lanes 1-2, φ6 virus s⁺ RNA segment (T7 transcript of pLM659 cut with XbaI; (Gottlieb et al., 1992)); lanes 3-4, COT-1 mRNA fragment (T7 transcript of a PCR fragment derived from pOY18 (Yarden et al., 1992) using the primers 5′-GTAATACGACTCACTATAGGCCGTGGTGGT-3′ (SEQ ID NO:5) and 5′-TTTTCTGAATTCTCTTGCCGCTTTTATTCT-3′ (SEQ ID NO:6); lanes 5-6, VMA-2 mRNA (T7 transcript of pRB30 (Bowman et al., 1988) cut with BamHI); lanes 7-8, VMA-1 mRNA (T7 transcript of pRB34 (Bowman et al., 1988) cut with NotI); lanes 9-10, VMA-1 antisense RNA (T3 transcript of pRB34 cut with EcoRV); lanes 11-12, PEP-4 mRNA (T3 transcript of pep4cDNA plasmid cut with Asp718) lanes 13-14, PEP-4 antisense RNA (T7 transcript of pep4cDNA cut with BamHI); lanes 15-16, antisense RNA of CCG-2 gene (T3 transcript of pLWdK1 (Bell-Pedersen et al., 1992) cut with Asp718). COT-1, VMA-1, VMA-2, PEP-4 and CCG-2 are N. crassa specific genes, and the relevant plasmids were obtained from FGSC (Kansas City, USA). (B) QDE-1 reactions done as in (A) but in the presence of [γ-³²P]GTP were separated by 16% denaturing PAGE. Lanes: 1, φ6 s+ RNA; 2, COT-1 mRNA fragment; 3, VMA-2 mRNA; 4, VMA-1 mRNA; 5, VMA-1 antisense RNA; 6, PEP-4 mRNA; 7, PEP-4 antisense RNA; 8, CCG-2 antisense RNA; 9, luc RNA. The sizes of ssRNA markers are shown on the right.

FIG. 7 demonstrates that QDE-1 initiates the synthesis of long RNA products preferably at the template 3′ terminal nucleotide.

(A) Flow chart for the assay carried out to determine the QDE-1 initiation site. RdRP reactions were carried out using 5′Δmₛ⁺ template (90 μg/ml; also see FIG. 3C) with no labeled nucleotides in the presence of either 20 μg/ml QDE-1, 20 μg/ml φ6Pol or an equal volume of M-200 buffer. G-50 purified reaction products were denatured by boiling for 1 min in 40% formamide and passed immediately through S-400 spin columns equilibrated with 20% formamide (Amersham Biosciences) to remove sRNAs. The flow-through fraction containing long RNAs was hybridized with the labeled primer specific to the antisense copy of 5′Δmₛ⁺. The primer extension was carried out with AMV-RT as described (Makeyev and Bamford, 2000) and the extended products were separated by 7% PAGE under denaturing conditions. (B) Radioactivity profiles of the PAGE separated products from (A) were plotted using phosphoimaging. The upper graph shows such profiles for the QDE-1 (black line) and M-200 buffer (gray line); the middle graph is for φ6Pol reaction products; the lower graph (T7 RNAP) shows primer extension done on sR5 RNA whose 5′ end is complementary to the 3′ end of 5′Δmₛ⁺. Positions corresponding to the template 3′-terminal sequences are indicated on each panel. The data show that QDE-1 prefers 3′-ultimate template nucleotide for initiating RNA synthesis, although other initiation positions may be used less frequently. As expected, φ6Pol and T7 RNA polymerase initiate RNA synthesis precisely, at a defined position (the 3′-most nucleotide in the φ6Pol case; see also (Makeyev and Bamford, 2000). (C) Small aliquots of the RdRP reactions carried out as in (A) were incubated in the presence of [α-³²P]UTP and analyzed by formaldehyde-containing agarose gel electrophoresis. Only template-length (1×; de novo initiated) and double template-length (2×; produced through “back-priming”) products appear in the case of φ6Pol, whereas sRNAs are produced by QDE-1 in addition to these two products. Peaks in (B) correspond to the 1× products.

FIG. 8 depicts the effect of spermidine on the sRNA size distribution.

QDE-1 was incubated at 30° C. for 1 h in the mixture containing luc RNA template and [γ-³²P]GTP in the absence (gray line) or presence (black line) of 1 mM spermidine. Reaction products were separated by 16% PAGE under denaturing conditions and the sRNA profiles were determined using a phosphoimager. Note that spermidine shifts the sRNA size distribution towards shorter products, having virtually no effect on the overall efficiency of the sRNA synthesis. Polyamines are known to stabilize RNA spatial structure (e.g. (Hanna and Szostak, 1994; Quigley et al., 1978), which suggests that QDE-1 might selectively recognize exposed single-stranded segments of RNA template and copy them into sRNAs, whereas the addition of spermidine reduces the effective length of these elements.

FIG. 9 demonstrates that sRNAs are synthesized along the entire template length.

(A) sRNAs were synthesized with QDE-1 on luc mRNA, purified through agarose gel and used to probe immobilized target RNAs. (B) Diagram shows luciferase-specific target RNAs: luc, lucΔ1 (T7 transcript of pTZluc(-stop) cut with EcoRI; (Makeyev et al., 1996), lucΔ2 (T7 transcript of pTZluc(-stop) cut with EcoRV), lucΔ3 (T7 transcript of pEM54 cut with XhoI), and a-luc (T7 transcript of pGEMluc cut with BamHI; Promega). (C) Target RNAs as in (B) and φ6-specific sR5 RNA (T7 transcript of pLM659 cut with EcoRV; (Gottlieb et al., 1992) were separated by formaldehyde-containing 1.5% agarose gel electrophoresis, transferred to a membrane and probed with the luc-specific sRNAs. Upper panel, EtdBr-stained gel; lower panel, autoradiogram of the membrane after hybridization.

FIG. 10 depicts reactions with dsRNA and primed ssRNA templates.

(A) RdRP mixtures containing 200 μg/ml of φ6 genomic dsRNAs (segments L, M and S) or 100 μg/ml LA virus-like particle genomic dsRNA were incubated with 40 μg/ml QDE-1, QDE-1ΔN, or φ6Pol and analyzed by 1% native agarose gel electrophoresis. (B) Schematic and (C) actual results of primer extension carried out with 40 μg/ml of QDE-1 or φ6Pol, or 500 units/ml of AMV-RT, as described under Experimental Procedures. The arrow indicates the position of the 136 nt extended product.

FIG. 11 depicts integrated model for posttranscriptional gene silencing that emerges from the data presented in this invention.

Aberrant ssRNA (abRNAs) are converted into dsRNA triggers by cellular RdRP. Either full-length or short (9-21 bp) dsRNA fragments are produced. Long dsRNAs are cleaved by a Dicer-like nuclease into siRNAs, whereas short dsRNA elements are recognized by a RISC-like nuclease. The latter event can be preceded by the QDE-2 (AGO1/RDE-1) mediated transfer of sRNAs to the cognate mRNAs. Some of the abRNA and mRNA degradation products might be used by the RdRP as efficient templates for the synthesis of secondary dsRNA triggers that will be diced up into siRNAs. Regardless of their origin, small ssRNAs act as guides for the RISC-catalyzed cleavage. This primer-independent scenario provides an alternative explanation for the RRF-1 mediated synthesis of secondary dsRNA triggers in C. elegans (Sijen et al., 2001).

FIG. 12 depicts RNA synthesis in the presence of chemically modified NTPs.

(A) Reaction mixtures containing biotin-11-CTP (NEN) were incubated for 1 h at 30° C. RNA products were purified from unincorporated nucleotides by passing through gel-filtration spin columns equilibrated with water (AutoSeq G-50, Amersham). Aliquots from the flow-through fractions were diluted 3M NaCl, 10 mM NaOH and spotted onto a pre-wetted Hybond N+ membrane (Amersham). The membrane was blocked in 2×SSC, 2.5% BSA, 0.5% Tween 20 for 1 h at 37° C. and then incubated with a 1:1000 dilution of HRP-conjugated streptavidin (NEN) for 30 min at 28° C. The membrane was washed 4 times (10 min each wash) with 2×SSC, 0.5% Tween 20, and the membrane-bound HRP was detected using ECL (Pierce) according to the manufacturer's instructions. P, reaction containing 25 μg/ml QDE-1ΔN polymerase but no RNA; R, reaction containing 90 μg/ml luc RNA but no polymerase; PR1 and PR2, reactions containing both 25 μg/ml QDE-1ΔN and 90 μg/ml luc RNA. The upper row correspond to 5 μl and the lower one correspond to 0.5 μl of the original polymerization reaction. Reactions contained 1 mM each of ATP and GTP, 0.2 mM of UTP and either 0.15 mM (PR2) or 0.2 mM (P, R, PR1) of biotin-11-CTP. PR2 reaction additionally contained 0.05 mM of the unmodified CTP.

(B) Reactions were carried out as in (A) except coumarin-5-CTP label (NEN) was used instead of biotin-11-CTP. After the gel-filtration step, incorporation of the coumarin label into RNA products was scored using fluorometry with the excitation wavelength of 402 nm. Shown are emission spectra of: P, reaction containing 25 μg/ml QDE-1ΔN polymerase but no RNA; R, reaction containing 90 μg/ml luc RNA but no polymerase; PR1, PR2 and PR3, reactions containing both 25 μg/ml ΔN and 90 μg/ml luc RNA. Reactions contained 1 mM each of ATP and GTP, 0.2 mM of UTP and 0.025 mM of coumarin-5-CTP. Unmodified CTP was added to reactions P, R, and PR3 to 0.2 mM, and to reaction PR2 to 0.05 mM.

FIG. 13 depicts RNA synthesis in the presence of different DNA templates.

(A) and (B) are, respectively, EtBr staining and autoradiogram of agarose gel separation of the reaction products under non-denaturing conditions. Reactions were programmed with the following templates: luc, luciferase mRNA; M13, circular ssDNA genome of bacteriophage M13; GEM, pGEM3Zf(+) plasmid linearized with HincII; GEM-b, pGEM3Zf(+) plasmid linearized with HincII and denatured by boiling for 2 min followed by chilling on ice. Where indicated, ΔN was added to the reaction mixtures to the final concentration of 25 μg/ml. M is the dsDNA marker lane.

(C) Autoradiogram of the heat-denatured QDE-1ΔN reaction products analyzed by agarose gel-electrophoresis. M^{ss} is the marker lane containing 1798 and 20 nt long labeled ssRNA.

FIG. 14 depicts a system where T7 RNA polymerase-directed transcription and ΔN-directed synthesis were carried out simultaneously in the same reaction vessel. Reactions were programmed with 50 ng/μl of plasmid pTZluc(-stop) (Makeyev et al., 1996) linearized with XhoI and carried out for 1 h under the conditions described in Example 2 for standard QDE-1 reactions with the difference that incubation temperature was 35° C. Products were analyzed by agarose gel electrophoresis. 0.25 μg of ΔN or/and 40 units of T7 RNA polymerase (Promega) was added per 10 μl of reaction mixture, as indicated on the top of the panels. M is dsDNA marker lane. Left panel, ethidium bromide staining; right panel, corresponding autoradiogram.

FIG. 15 depicts RNAi experiment where QDE-1 reaction products were used to induce gene-specific silencing in C. elegans.

QDE-1 reaction products are capable of inducing RNAi in C. elegans. Shown are representative images of the F1 progeny of the hermaphrodites treated with either GFP-specific dsRNA products of QDE-1 (a-b) or GFP ssRNA (c-d). Photographs in (a) and (c) were taken using Hoffman modulation contrast; (b) and (d) are corresponding fluorescent images. Note that two of the three worms in (a-b) show no detectable GFP fluorescence. Similar results were obtained using ΔN polymerase reaction products (not shown.)

### DETAILED DESCRIPTION OF THE INVENTION

The present invention provides a method for producing short complementary RNA copies of RNA and DNA templates scattered along the entire template length by using RNA polymerase from the RNA silencing pathway, an enzyme that has never been isolated or characterized before. It is an advantage of this invention that said polymerase from the RNA silencing pathway and derivatives thereof are provided in a soluble enzymatically active form. “RNA silencing” is a generic term used here to refer to several related phenomena found in eukaryotic cells such as postranscriptional gene silencing, co-suppression, virus-induced gene silencing, RNA interference (RNAi) etc. A general feature of all these phenomena lies in sequence-specific degradation of target RNAs by RNA trigger molecules (see BACKGROUND OF THE INVENTION for more detail). A polymerase protein of the present invention originates from a eukaryotic cell or has the amino acid sequence of such a cellular polymerase or is a derivative of such a polymerase.

For the purpose of this invention, the disclosed RNA polymerase is variously referred to as “polymerase protein”, “polymerase”, “cellular RdRP” or simply “RdRP” or even protein or polypeptide. The invention provides the first direct evidence that an isolated polymerase of the RNA silencing pathway is capable of RNA synthesis in vitro when contacted with RNA or DNA templates under suitable conditions.

1. Objects of this Invention

This invention provides a method for producing a nucleic acid product, comprising that the polymerase protein of this invention is contacted with a nucleic acid template under conditions sufficient for the function of the enzyme.

It is characteristic for the polymerase protein of this invention that it is capable of producing short complementary RNA copies of the nucleic acid template, which copies are scattered throughout the entire template length and, optionally, template-length complementary RNA copies. In other words the polymerase protein is capable of synthesizing noncontiguous RNA-strands complementary to the template in addition to template-length copies. This is a totally new feature for an RNA polymerase.

By short RNA copies or strands are here meant copies the length of which is 7 to 40 nt, mostly 9 to 21 nt. The ratio of the nucleic acid products comprising short RNAs and the nucleic acid products comprising template-length (long) RNAs can be adjusted by the reaction conditions. More specifically the ratio can be adjusted by the ionic concentration, such as by suitable concentration of divalent metal ions (such as Mn²⁺ and Ca²⁺). Also the length of the noncontiguous or short RNAs can be adjusted towards shorter RNAs by certain chemicals, such as spermidine.

The polymerase protein of the invention can catalyze RNA synthesis using single-stranded RNA or single-stranded DNA templates and the RNA or DNA template can be linear or circular.

The produced RNA strands are usually annealed to the template but they can be also denatured from the template.

The polymerase of this invention may originate from a eukaryotic cell. Specifically it may originate from an organism selected from the kingdoms of Fungi, Viridiplantae, Metazoa, or the group of Mycetozoa.

More specifically the polymerase of this invention may originate from an organism selected from the subset of genera Neurospora, Arabidopsis, Caenorhabditis, and Dictyostelium, preferably organisms Neurospora crassa, Arabidopsis thaliana, Caenorhabditis elegans, and Dictyostelium discoideum. In particular, the polymerase is QDE-1 protein of Neurospora crassa or an altered or a genetically modified derivative of QDE-1.

The RNA polymerase of this invention, which is capable of producing short complementary RNA copies of the nucleic acid template, which copies are scattered throughout the entire template length and, optionally, template-length complementary RNA copies, is encoded by a nucleic acid sequence selected from the group of:
(a) a nucleic acid sequence, which in addition to optional tag sequence comprises the sequences of SEQ ID NO: 1, corresponding to QDE-1 protein, or SEQ ID NO: 3, corresponding to ΔN, a truncated version of QDE-1;
(b) a nucleic acid sequence encoding a polypeptide, which in addition to optional tag sequence comprises the sequences of SEQ ID NO: 2, corresponding to QDE-1 protein, or SEQ ID NO: 4, corresponding to ΔN;
(c) a nucleic acid sequence, which differs from the nucleic acid sequence of (a) or (b) due to degeneracy of the genetic code;
(d) a nucleic acid sequence hybridizing to the nucleic acid sequence of (a), (b) and/or (c) under stringent conditions; and
(e) a nucleic acid sequence encoding a polypeptide comprising the amino acids 709 to 1402 of SEQ ID NO:4 or any sequence longer than that up to the sequence having the amino acids 2 to 1402 of SEQ ID NO: 2; and
(f) a nucleic acid sequence encoding an amino acid sequence, which shows at least 50% identity to the amino acid sequence of SEQ ID NO: 2.

An optional tag sequence is presented in the Sequence Listing as SEQ ID NO: 14 (nucleic acid sequence) and SEQ ID NO:15 (amino acid sequence).

This invention also provides an RNA polymerase form, which has an improved solubility, which results in higher yields of the active polymerase. More specifically this invention provides an isolated polypeptide, which has sufficient RNA polymerase activity, which has enhanced solubility resulting in at least 3 times higher yield of the active polymerase, than in the case of polypeptide comprising the amino acid sequence of SEQ ID NO: 2 or encoded by the nucleic acid sequence comprising SEQ ID NO: 1; and which is encoded by a nucleic acid sequence selected from the group of:
(a) a nucleic acid sequence comprising the nucleic acid sequence of SEQ ID NO: 3, which encodes ΔN;
(b) a nucleic acid sequence encoding a polypeptide comprising the amino acid sequence of SEQ ID NO: 4, which has the amino acid sequence of ΔN;
(c) a nucleic acid sequence, which differs from the nucleic acid sequence of (a) or (b) due to degeneracy of the genetic code;
(d) a nucleic acid sequence hybridizing to the nucleic acid sequence of (a), (b) and/or (c) under stringent conditions; and
(e) a nucleic acid sequence encoding a polypeptide comprising the amino acids 709 to 1402 of SEQ ID NO:4 or any sequence longer than that up to the sequence having the amino acids 2 to 1402 of SEQ ID NO: 2; and
(f) a nucleic acid sequence encoding an amino acid sequence, which shows at least 50% identity to the amino acid sequence SEQ ID NO: 2.

By sufficient RNA polymerase activity is meant here a measurable activity of the polymerase to produce short and long RNA copies of a nucleic acid template as described above. The activity is defined to be measurable if the polymerase protein is contacted with nucleic acid template under conditions sufficient for the function of the enzyme and the nucleic acid products can be detected by ethidium bromide (EtBr) staining.

The yields obtained by a nucleic acid sequence encoding the shortened forms of the RNA polymerase of this invention are higher compared to the yields obtained by the full length sequence, since the product is in soluble and active form. In other words, the measured amount of active protein is higher obtained by nucleic acid sequences encoding the shortened forms of the RNA polymerase than obtained by the full length sequence. The full-length sequence produces protein mainly in aggregated form and the measured amount of the soluble protein is therefore lower. Preferably the yield of the soluble protein obtained by the shortened forms of the RNA polymerase are 3 times higher, more preferably 5 times higher, and most preferably they are 10 times higher than obtained by the full length sequence of the RNA polymerase protein.

The most preferred shortened form of the RNA polymerase of this invention is ΔN comprising the amino acids 377 to 1402 of SEQ ID NO:2. However, enhanced yields of RNA polymerase of this invention can be obtained also by nucleic acid sequences encoding a longer amino acid sequence than ΔN, i.e. a sequence which is shorter than the full length sequence having 1402 amino acids, but which comprises amino acids from the N-terminal part of SEQ ID NO:2. The shortened form can also be shorter than ΔN, i.e. nucleic acid sequences encoding amino acid sequences longer than from amino acid 709 to amino acid 1402. Cogoni et at. 1999 discloses the cDNA encoding the full length QDE-1 and the amino acid sequence encoding a fragment of 570 amino acids of QDE-1 extending from amino acid 710 to amino acid 1280. This shortened sequence did not in our experiments produce active and soluble RNA polymerase and therefore it is not the polypeptide which could be used in this invention. The full-length amino acid sequence of QDE-1 can be used in the methods of this invention, but a preferred form of the RNA polymerase of this invention is a soluble polypeptide or protein of this invention.

The present invention relates also to nucleic acid sequences, which differ from SEQ ID NO:1 or SEQ ID NO:3 or from the sequences encoding SEQ ID NO: 2 or SEQ ID NO: 4 due to degeneracy of the genetic code. The present invention relates furthermore to nucleic acid sequences, which hybridize to the SEQ ID NO:1 SEQ ID NO:3 or to the sequences encoding SEQ ID NO:2 or SEQ ID NO:4 or to the degenerated sequences under conventional hybridization conditions, preferably under stringent conditions such as described by Sambrook and Russell (2001). High stringency hybridization may be between about 65° C. and 70° C. in a solution of 6×SSC, 0.5% SDS, 5× Denhardt's solution and 100 μg of non-specific carrier DNA. The preferred probe is SEQ ID NO:3, which encodes ΔN. Excess probe is removed by washing in a solution having the equivalent in ionic strength of less than about 0.2× to 0.1× SSC. A typical high stringency Wash is twice for 30 minutes at 55° C. and three times for 15 minutes at 60° C.

These nucleic acid sequences that hybridize to the nucleic acid sequences of the present invention can in principle be derived from any organism possessing such nucleic acid sequences. Preferably, they are derived from eucaryotes as desrcibed here earlier. Nucleic acid sequences hybridizing to the nucleic acid sequences of the present invention can be isolated, e.g., from genomic libraries of various organisms.

Such nucleic acid sequences can be identified and isolated by using the nucleic acid sequences of the present invention or fragments of these sequences or the reverse complements of these molecules, e.g. by hybridization according to standard techniques (see Sambrook and Russell 2001).

As hybridization probes, one can use nucleic acid molecules that have exactly or substantially the same nucleotide sequence as SEQ ID NO:1 or fragments of said sequence. Preferably the nucleotide sequence SEQ ID NO:3 is used. The fragments used as hybridization probes can also be synthetic fragments obtained by conventional synthesis techniques, the sequence of which is substantially identical to that of the nucleic acid sequences of the invention. Once genes hybridizing to the nucleic acid sequences of the invention have been identified and isolated, it is necessary to determine the sequence and to analyze the properties of the proteins coded for by said sequence.

The term “hybridizing nucleic acid sequence” includes fragments, derivatives and allelic variants of SEQ ID NO:1 or SEQ ID NO:3 encoding an identical or substantially similar protein or a biologically active fragment thereof. Fragments are understood to be parts of nucleic acid sequences long enough to code for the described protein (or substantially similar protein) or a biologically active fragment thereof. The term “derivative” means in this context that the nucleotide sequences of these molecules differ from the sequences of the above-described nucleic acid molecules in one or more positions and are highly homologous to said sequence.

“% Identity” means here percentage of identical amino acids being present at corresponding positions when two amino acid sequences are aligned to give the maximal amount of identical nucleotides or amino acids at corresponding positions. This invention relates to proteins, the amino acid sequence of which has at least 50%, preferably at least 60%, more preferably at least 70%, still more preferably at least 80%, even more preferably at least 90%, and most preferably at least 95% identity at the amino acid level to the specific amino acid sequence of SEQ ID NO:2. The identity % can be calculated by the formula:
(Number of identical residues)*100% /(length of the longer sequence SEQ ID NO:2=1402)=identity percentage

Protein engineering can be used to construct modified polymerases possessing improved properties. Such modifications may include, for example, mutating amino acid sequence of QDE-1 polymerase or a fragment of it or a protein with substantially similar properties in order to make said polymerase or protein less template-specific, more (or less) processive, or optimize the enzyme for primer extension, sequencing, amplification of nucleic acids, etc.

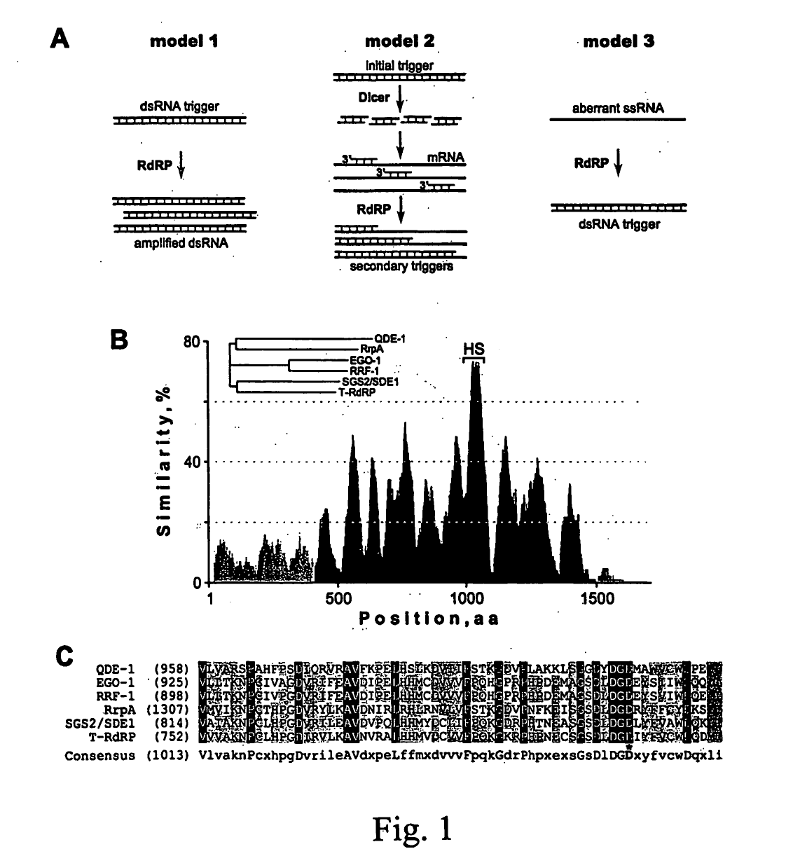

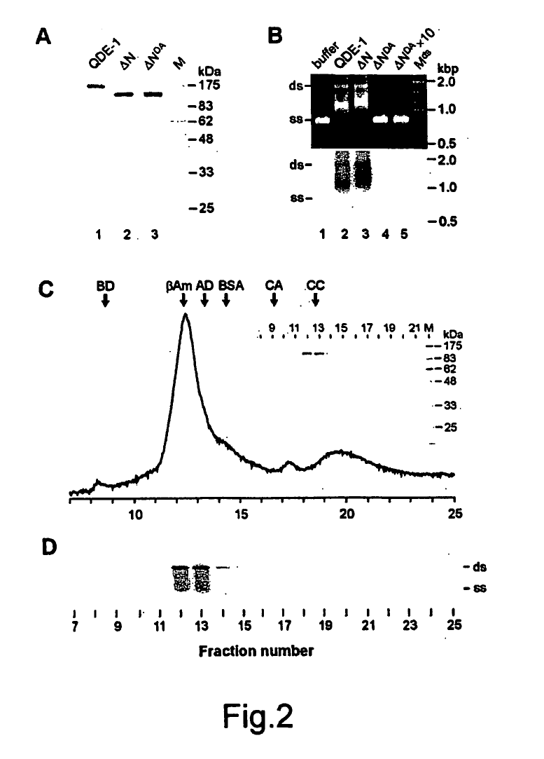

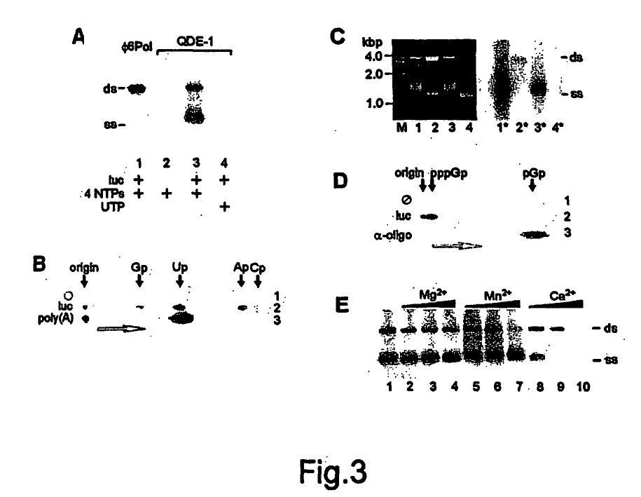

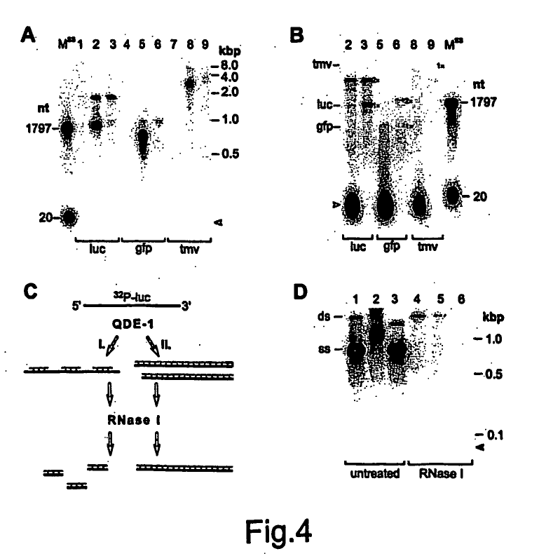

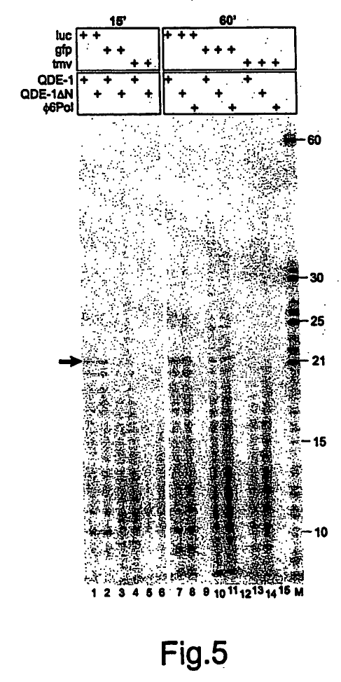

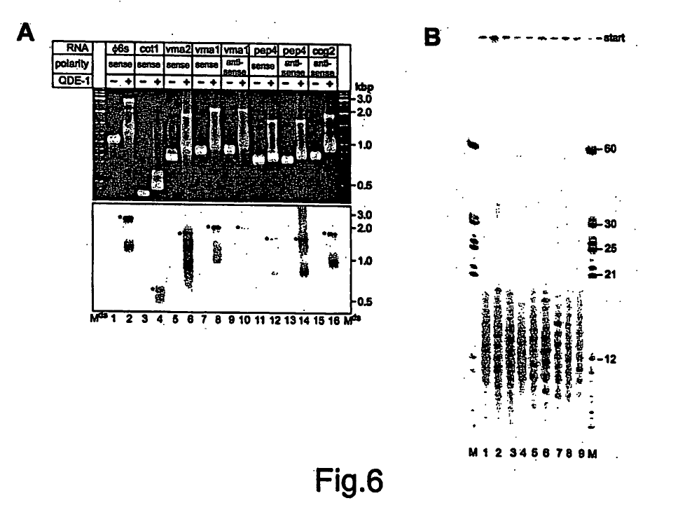

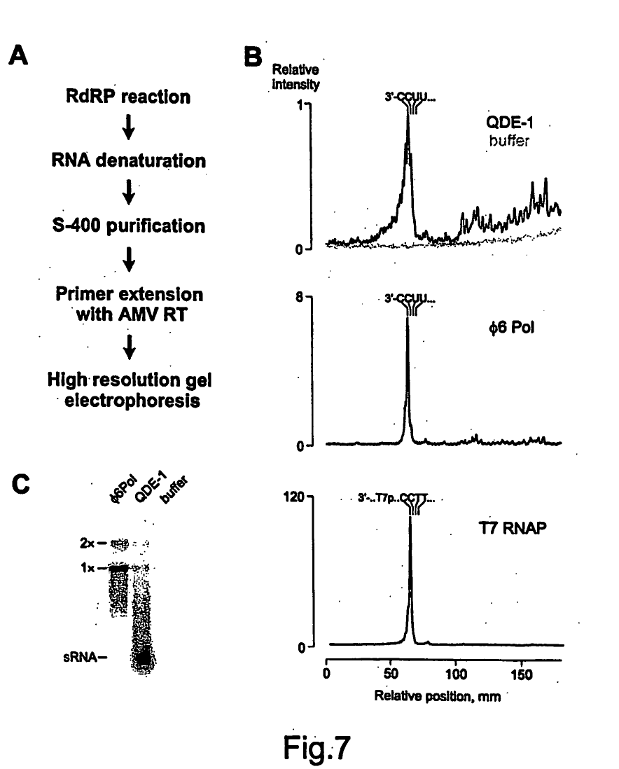

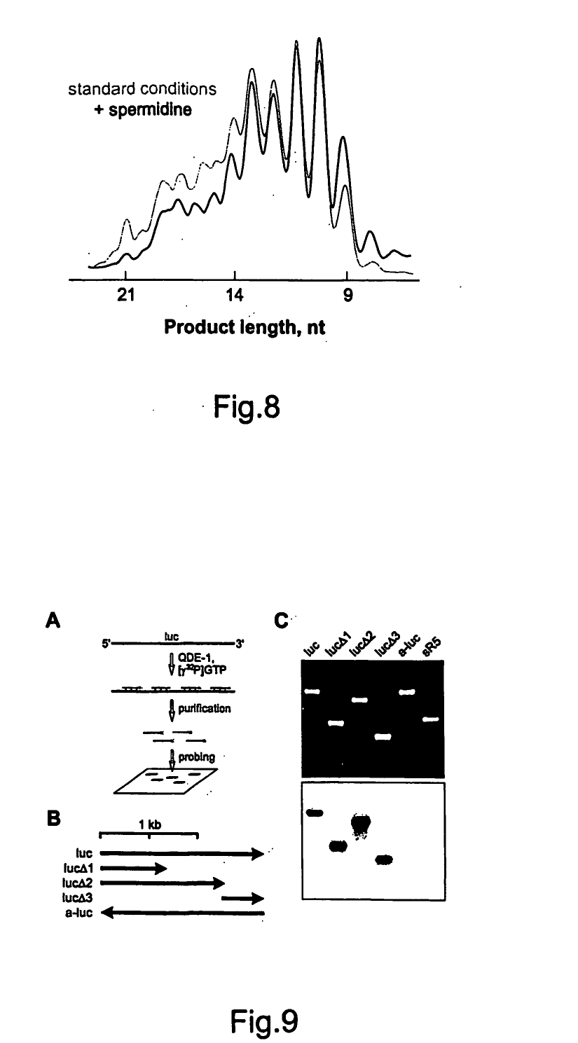

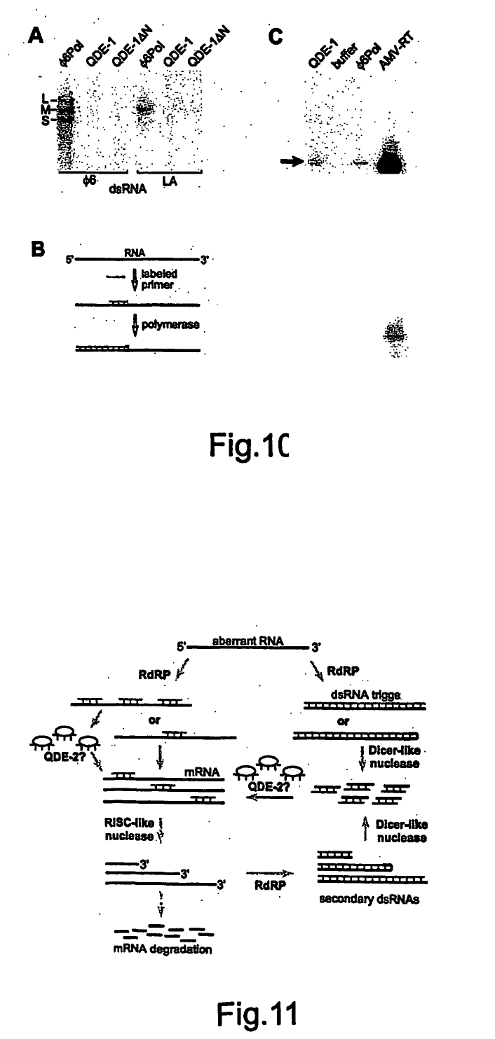

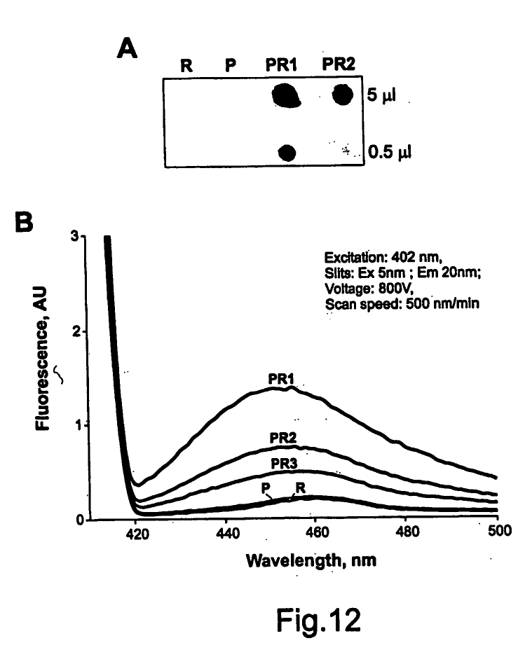

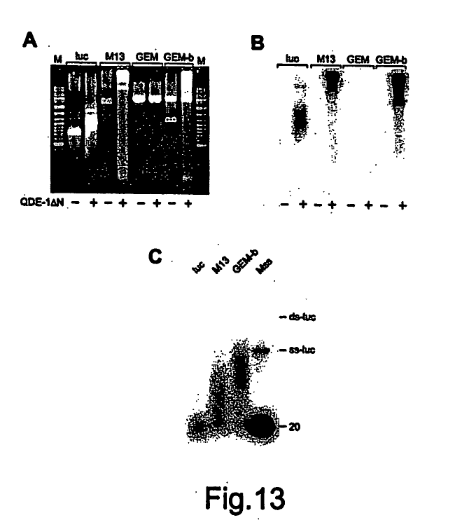

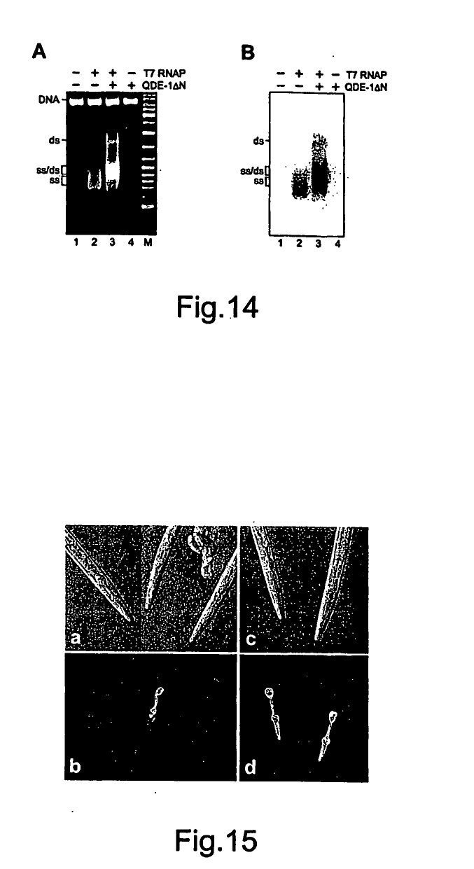

In an embodiment of this art, several modified versions of QDE-1 were generated and characterized (FIG. 2A). One of the modified proteins, designated ΔN, QDE-1 N-terminal part was deleted. The protein retains enzymatic activity of the full-length QDE-1 and also has two additional bonuses: (1) the yield of purified ΔN is higher than QDE-1 and (2) ΔN is more stable than QDE-1 when stored at +4° C. (not shown). Yet another modified protein was ΔN with a point mutation converting its Asp1011 residue (numbered for the full-length QDE-1) into Ala. This protein (ΔN^{DA}) was completely inactive as a polymerase, since Asp1011 is likely involved in the catalysis of the nucleotidyl-transferase reaction as based on the sequence conservation (FIG. 1B). However, the mutation does not affect the overall fold of ΔN which is apparent from native gel-filtration analysis (see FIGS. 3C and D; and not shown).

In addition to the QDE-1 derivatives described above, two spontaneous QDE-1 mutants were obtained in a purified form and characterized. These were full-length QDE-1 proteins containing either one amino acid change (H1161Y) or four amino acid changes (M131T, D893G, A1000T, F1124L). Interestingly, both mutants retained detectable RNA polymerization activity, being able to produce both full-length and short RNA products.

The invention also concerns purified RdRP proteins of RNA silencing pathway, which are derived from organisms other than N. crassa. FIG. 1B lists several RdRP-like proteins that are involved in PTGS according to genetic data. Given their sequence similarity to QDE-1, which is shown here to possess polymerase activity, it is expected that at least some of these other proteins can also catalyze RNA synthesis after being provided in a purified form.

This invention provides also a nucleic acid sequence, which encodes the polypeptide of this invention as well as a vector, which comprises the said nucleic acid sequence operationally linked with regulatory sequences required for gene expression and a host cell comprising the said vector.

This invention furthermore provides a method for producing a polymerase protein, which comprises culturing the host cell under conditions suitable for the expression of the protein. The protein can be recovered from the host cell or culture medium and optionally purified.

This invention provides a method for producing RNA in vitro, comprising the steps of:
(a) providing ssRNA or ssDNA template;
(b) contacting said ssRNA template with the protein or polypeptide of this invention under conditions sufficient for RNA synthesis.

The reaction mixture can be used as such in subsequent reactions or the newly produced RNA species can be recovered from the reaction mixture.

In the above method steps (a) and (b) can be carried out at the same time or sequentially in the same reaction vessel.

When single-stranded RNA is used as template the ssRNA template is provided by transcribing a DNA template with a DNA-dependent RNA polymerase. Examples of suitable DNA-dependent RNA polymerases are preferably derived from a bacteriophage selected from the group of T7, T3, and SP6 bacteriophages.

The newly produced RNA strands are annealed with the template to form dsRNA elements or, alternatively, are denatured from the template.

In the method RNA synthesis can be initiated without a primer, but RNA synthesis can be also initiated from the 3′ end of a nucleic acid primer complementary to the RNA or DNA template.

The reaction mixture for RNA synthesis comprises at least one nucleoside triphosphate optionally labeled with a radioactive isotope or is chemically modified, pH buffer, ammonium acetate, PEG, Mg²⁺-ions, Mn²⁺-ions and/or non-ionic detergent. The method can specifically be used for producing radioactively or chemically labeled RNA probes. The method may comprise also a step of purifying the newly produced labeled RNA from the components of the reaction mixture. After the purification step the labeled RNA species can be used as probes for Southern or Northern blot analyses. The labeled RNAs can be used also as probes for a fluorescent in situ hybridization analysis or as probes for a microarray analysis.

The polypeptide or protein of this invention can be used for various applications, such as for studying nucleic acid structure, for studying nucleic-acid protein interactions and for producing RNA trigger molecules to induce RNA interference in vivo or in vitro. This invention therefore also provides a method for studying nucleic acid secondary structure, preferably RNA secondary structure. The method comprises the steps of:
(a) providing nucleic acid target molecule, preferably RNA target molecule;
(b) contacting said target molecule with the protein or polypeptide of the invention under conditions sufficient for RNA synthesis in a mixture additionally comprising radioactively or chemically labeled nucleotides, so that single-stranded elements of said target RNA are copied by the polymerase of this invention;
(c) recovering and optionally purifying the newly produced labeled nucleic acid species from the reaction mixture;
(d) using said labeled nucleic acid species as probes for microarray chip that comprises nucleic acid fragments of said target molecule;
(e) interpreting data from the microarray analysis to deduce which parts of the target molecule are single-stranded; and optionally
(f) building a model for the secondary or tertiary structure of the target molecule.

Further, this invention provides a method for studying nucleic acid-protein interactions, preferably RNA-protein interactions. The method comprises the steps of:
(a) providing a nucleic acid target and nucleic acid binding protein, preferably an RNA target and an RNA-binding protein;
(b) contacting said target and the solution of said protein in an experimental mixture under conditions sufficient for target-protein interaction, and in a separate vessel, contacting said target with a control solution that lacks said protein.
(c) contacting said experimental and control mixtures with the protein or polypeptide of the invention under conditions sufficient for RNA synthesis;
(d) recovering and optionally purifying the newly produced labeled nucleic acid species from both reaction mixtures;
(e) using the two sets of labeled nucleic acid species as probes for two identical microarray chips that comprise nucleic acid fragments of the target;
(f) interpreting data from the two microarray analyses to deduce which parts of the target molecule are accessible for the RNA synthesis;
(g) comparing the two data sets to determine the difference between target in experimental and control mixtures; and optionally
(h) interpreting the difference between the two data sets as a model for nucleic acid-protein interactions.

Furthermore, this invention provides a method for producing RNA trigger molecules to induce RNA interference in vivo or in vitro. The method comprises the steps of:
(a) providing RNA or DNA template;
(b) contacting said RNA or DNA template with the protein or polypeptide of the invention under conditions sufficient for RNA synthesis in a mixture comprising: nucleic acid template, protein of this invention, nucleoside triphosphates, and optionally pH buffer, ammonium acetate, PEG, Mg²⁺ ions, Mn²⁺ ions and/or non-ionic detergent; and
(c) incubating the reaction mixture at temperature sufficient for RNA synthesis.

In the method, said RNA or DNA template may originate from a cell or a virus. Said RNA template can be obtained by transcribing a DNA template with a DNA-dependent RNA polymerase, preferably derived from a bacteriophage selected from the group of T7, T3, and SP6 bacteriophages.

In the method, steps (a) and (b) can be carried out at the same time or sequentially in the same reaction vessel.

This invention provides also a kit comprising the protein or polypeptide of this invention. The kit may further comprise additives necessary for a detectable level of RNA synthesis. The kit may comprise, for example, nucleoside triphosphates in concentrations sufficient for RNA synthesis, at least one nucleoside triphosphate labeled with a radioactive isotope or modified chemically and/or a standard nucleic acid preparation (or preparations) with characterized capacity to serve as a template (templates) for RNA synthesis by the protein or polypeptide of the invention.

It is a major advantage of this invention that said soluble polymerase is obtained from a recombinant source, because this ensures high protein yields and also makes it possible to alter polymerase properties using molecular approaches. Preferred polymerases of the invention, QDE-1 protein originating from fungus Neurospora crassa, and its genetically altered forms, can use a number of RNA templates in vitro generating two types of daughter RNA chains: short (7 to 40 nt, preferably 9-21 nt) and long (up to the template length) ones. The polymerase protein does not require a primer for the initiation of RNA synthesis, although it also is able to initiate RNA synthesis from a primer.

The efficiency of RNA synthesis by QDE1, ΔN and some of their derivatives is sufficiently high, thus suggesting that the RNA products can be used, advantageously, for many practical applications, such as e.g. producing RNA probes for hybridization-based techniques or RNA triggers for inducing RNA silencing in living organisms.

2. Preparation of the Polymerase Protein of this Invention

This invention provides a method of expression and purification of the protein of this invention, preferably QDE-1 protein of N. crassa or QDE-1 genetic derivatives. The method comprises the steps of:
(a) culturing cells containing nucleic acid with a sequence encoding a polymerase protein of this invention to express said protein from said nucleic acid;
(b) recovering the protein from the host or from the culture medium; and
(c) purifying said protein using at least one chromatography step.

The nucleic acid sequences of this invention may be operably linked to the regulatory elements in an expression vector, which is introduced into a chosen host cell to produce the protein of this invention. Expression of the polymerase of this art may be achieved in any suitable host cell, e.g., animal, plant, fungal or bacterial cell. In the currently preferred embodiment of this invention, expression host is baker's yeast Saccharomyces cerevisiae.

Upon its synthesis in the host cell, the protein is preferably isolated and purified by the steps, comprising:
(a) disrupting the host cells in a buffer to obtain a cell lyzate;
(b) clarifying said lyzate by centrifugation;
(c) purifying the protein using at least one step of chromatography, preferably immobilized metal-affinity chromatography to obtain a fraction that is essentially free of nuclease and protease activities.

“Essentially free of nucleases and proteases” means here that the purified protein preparation does not contain a detectable amount of nucleases and/or proteases.

As specific embodiments, the expression and purification of QDE-1 polymerase and its enzymatically active N-terminally truncated version (ΔN) are described in Example 1.

3. Reaction Conditions

In addition to the methods for expression and purification of a cellular RdRP, this invention also concerns method and kit for in vitro RNA synthesis.

In its general form the method for RNA synthesis comprises the following steps:
(a) providing RNA or DNA template;
(b) contacting said template with the protein of the invention under conditions sufficient for RNA synthesis; and optionally
(c) recovering the newly produced nucleic acid species from the reaction mixture.

According to a specific embodiment of this invention, the mixture for the RNA synthesis contains components listed in Example 2. The reaction mixture is incubated at 30° C. for 1 h. Somewhat altered conditions can also support a detectable level of RNA synthesis. Specifically, modified conditions may imply one or several changes selected from the group:
(1) a different final concentration of the protein of this invention in the reaction mixture (preferably 0.1 to 200 μg/ml);
(2) a different concentration of nucleoside triphosphates (preferably 0.1 to 4 mM of each NTP);
(3) a different concentration of MnCl₂, preferably 0 to 4 mM;
(4) a different temperature of incubation (preferably 15 to 42° C.).
   3.1. RNA Templates

Currently preferred method for RNA synthesis comprises the steps of:
(a) providing single-stranded RNA template, preferably at a final concentration of 40-400 μg/ml;
(b) contacting said RNA template with the protein of the invention (preferably 1 to 100 μg/ml) under conditions sufficient for RNA synthesis in a mixture containing additionally pH buffer (preferably HEPES pH 7.8), Mg²⁺ ions (preferably 5-10 mM), nucleoside triphosphates (preferably 0.2 to 1 mM of each NTP), and, optionally, Mn²⁺ ions (preferably up to 2 mM) and nonionic detergent, preferably 0.1 to 0.2% of Triton X-100);
(c) incubating the reaction mixture at a permissive temperature, preferably 20 to 37° C., and optionally
(d) recovering the newly produced nucleic acid species from the reaction mixture.

It is highly advantageous that the polymerases of this invention, most preferably ΔN and derivatives of QDE-1, accept a wide range of ssRNA templates converting all or a substantial part of input RNA into double-stranded form (see Example 2 and relevant figures for details). It is also advantageous that two distinct types of reaction products are generated regardless of the template sequence: (1) long RNA copies close or equal to the template length and (2) short RNA oligonucleotides (called sRNAs, for the purpose of this invention), 7 to 40 nt, mostly of 9 to 21 nucleotide long that are scattered along the template length (see Example 2 and relevant Figures for details). Under currently preferred conditions, both types of RNA products remain attached to their encoding template to form dsRNA elements. Based on the dual polymerization mechanism, we propose a novel model for the role of RdRP of this invention in postranscriptional gene silencing (FIG. 11).

Importantly, choosing appropriate conditions it is possible to modulate the ratio between the newly produced long and short RNA copies. In a specific example, concentration of divalent metal ions (such as Mn²⁺ and Ca²⁺) could change the relative amount of the two different types of reaction products (FIG. 3E). Furthermore, reaction conditions can also modulate the length distribution of the short RNAs produced on a given RNA template. FIG. 8 demonstrates that, in the presently preferred embodiment, the addition of polyamine spermidine shifts the short RNA distribution towards shorter species.

In a specific embodiment of the present invention, ssRNA template for the QDE-1-catalysed RNA synthesis can be provided by transcribing DNA templates with a DNA-dependent RNA polymerase. Preferably, the DNA-dependent RNA polymerase is derived from a bacteriophage. It is most advantageous that the bacteriophage is selected from the group consisting of T7, T3, and SP6 bacteriophages. In some embodiments of the art, said transcribing a DNA template with a DNA-dependent RNA polymerase and QDE-1-catalyzed replicating the newly produced linear ssRNA can occur in the same reaction vessel. Special experiments were carried out in order to demonstrate the possibility of the latter approach (see FIG. 14). Linear dsDNA containing promoter for T7 RNA polymerase (namely, pTZluc(-stop) cut with XhoI) was incubated with both T7 RNA polymerase and the N-terminally truncated version of QDE-1 polymerase (ΔN) at 35° C. The reaction products comprised essentially the mixture of ssRNA and different forms of dsRNA in the case both polymerases were added to the reaction mixture, whereas only ssRNA was formed if only T7 RNA polymerase was present.

In the presently preferred embodiments, intact dsRNA templates, such as genomic segments of dsRNA, viruses cannot be used by the polymerase of this invention to direct detectable RNA synthesis (FIG. 10A). However, dsRNA that are denatured before reaction for example by boiling for 1 min can be accepted as templates efficiently (not shown).

In a specific embodiment of this invention, isolated QDE-1 and ΔN polymerases can initiate RNA synthesis from the 3′ end of a complementary oligonucleotide annealed to an RNA template (FIGS. 10B, 10C and not shown). This primed RNA synthesis can be of future interest for certain practical applications, such as primer extension methods.

3.2. DNA Templates

According to some additional embodiments of the art, a set of single-stranded DNAs (M13 phage linear ssDNA or linerized and heat-denatured plasmid DNA) was shown to be replicable with QDE-1 and ΔN under similar conditions as described above for single-stranded RNA (FIG. 13 and not shown). The reaction results in duplexes consisting of a template DNA and a newly produced RNA replica Therefore, this invention relates to a method for producing RNA in vitro, comprising the steps of:
(a) providing DNA template in effectively single-stranded form;
(b) contacting said DNA template with the protein of the invention under conditions sufficient for RNA synthesis; and optionally
(c) recovering the newly produced DNA-RNA hybrid products from the reaction mixture.

The DNA-programmed reactions can be of potential utility for the methods that require transcription of DNA sequence into the RNA form.

4. Practical Applications of the Polymerase of this Invention

Based on the findings above, the present invention provides methods for producing RNA using polymerase of this invention contacted with different nucleic acid templates. Some of these methods are specifically suited for some downstream applications: such as producing interfering RNAs and radioactively or chemically labeled RNAs that can be used as probes.

4.1. RNA Interference

This invention relates to a method for producing RNA capable of inducing RNA interference (RNAi) in animals and protozoa and related phenomena known as RNA silencing in other eukaryotic organisms. The method comprises the steps of:
(a) providing single-stranded RNA template;
(b) contacting said single-stranded RNA with the protein of the invention under conditions sufficient for RNA synthesis in order to convert at least part of the single-stranded nucleic acid template into a product comprising double-stranded elements;
(c) recovering said product from the reaction mixture in a sufficiently pure form;
(d) assaying the RNAi activity of said RNA products in vivo or in vitro using an appropriate eukaryotic system.

In a specific embodiment, said appropriate eukaryotic system is a live nematode Caenorhabditis elegans. For inducing RNAi, mRNA of GFP (green fluorescent protein) was first converted into dsRNA products using QDE-1 or ΔN. Recombinant C. elegans expressing GFP were soaked in solution containing said products and the gene silencing effect was scored in the F1 progeny as a percent of worms showing fluorescence below detection limit (FIG. 15). Other organisms that support RNA silencing mechanism can also be potential targets in other embodiments (as described in the BACKGROUND OF THIS INVENTION) and the ways of directing dsRNA products into organism or cell can also vary from soaking to injecting to using ballistic, lipofection or other delivery methods.

It is noteworthy that the methods described in the prior art of inducing RNAi usually rely on the synthesis of sense and antisense RNAs homologous to the gene to be silenced, subsequent purification of these two complementary RNAs, and their annealing so that to form a dsRNA molecule that can be used for RNAi experiments. The present invention offers the advantage that the dsRNA trigger can be generated simultaneously with the transcription of a gene of interest with a DNA-dependent RNA polymerase, as exemplified in FIG. 14. This coupled system is faster and less laborious than the sense-antisense annealing strategy and it would be especially welcomed for high-throughput RNAi methods, when thousands of different genes need to be silenced and the corresponding phenotypes determined (for more detail see Barstead, 2001; Fraser et al., 2000; Gonczy et al., 2000; Maeda et al., 2001).

In a specific embodiment of this invention dsRNA trigger molecules for inducing RNAi can be produced by the polymerase of this invention using effectively single-stranded DNA templates according to a method, which comprises the steps of:
(a) providing DNA template in a single-stranded form;
(b) contacting said DNA template with the protein of the invention under conditions sufficient for RNA synthesis;
(c) dissociating the newly produced RNA product from its encoding DNA template;
(d) in the same or new reaction mixture, contacting said dissociated RNA product with the protein of the invention under conditions sufficient for RNA synthesis so that dsRNA products are formed.
(e) recovering said dsRNA products from the reaction mixture in a sufficiently pure form;
(f) assaying the RNAi activity of said RNA products in vivo or in vitro using an appropriate eukaryotic system.
   4.2. Generation of Non-Radioactively Labeled RNAs

In addition to unlabeled or radioactively labeled nucleoside triphosphates, polymerases of the present invention can incorporate chemically modified nucleotides into the RNA product. This makes it possible to assay RNA synthesis using a non-radioactive methodology, such as that based on detecting fluorescence or chemiluminescence.

In a preferred embodiment, standard RNA polymerization mixture containing a ssRNA template was supplemented with coumarin-5-CTP or biotin-11-CTP. Reactions were incubated for 1 hour at 30° C. The reaction mixtures were then passed through gel-filtration spin columns to purify RNA products from the non-reacted nucleotide analogs and from the other low molecular weight contaminants. Incorporation of the nucleotide analogs into the newly produced RNA was then measured in the flow-through fractions using a spectrofluorometer (in the case of coumarin-5-CTP) or a dot blot assay (for biotin-11-CTP). In both cases, a detectable part of the modified nucleotide was incorporated into the RNA products (FIG. 12).

4.3. Using Labeled RNAs as Probes

It is also in the scope of this invention that labeled RNA products generated by purified cellular RdRPs can be used as RNA probes for downstream applications based on radioactive or non-radioactive detection. Examples of such applications include microarray technology, fluorescent in situ hybridization, and Northern and Southern blotting. It is of immense advantage for said applications that the RdRP of this invention can produce substantial amounts of short complementary sRNAs with the preferred length of 7-40 nt, mostly 9-21 nucleotides.

In a preferred embodiment of this invention, short RNA fraction of reaction products is purified from the template and long RNA products using a denaturing gel-electrophoresis (FIG. 9A). Other methods of purification are also possible such as for example purification using gel-filtration or ion-exchange chromatography under denaturing conditions. Anyhow, the recovered fraction of the short RNA is suitable for subsequent hybridization procedures as documented in FIG. 9C. It is of obvious benefit for the hybridization procedures that the polymerase of this invention generates short RNA scattered along entire template length (as concluded from FIG. 9).

4.4. Specialized Applications for Studying Nucleic Acid Structure and Nucleic Acid-Protein Interactions

As it appears from the experiment presented in FIG. 8, short RNAs are produced on the elements of template RNA that are effectively single-stranded, that is not involved in a stable secondary or tertiary structure. This property of the polymerase of this invention suggests a novel technique for gaining insights into secondary or/and tertiary structure of essentially any given ssRNA target (or ssDNA target). In the currently preferred version of this method includes the following steps:
(a) provided ssRNA target is first incubated with the polymerase of this invention under conditions sufficient for the synthesis of labeled sRNAs;
(b) labeled short RNAs are recovered from the reaction mixture and optionally purified from the template and long RNA products using e.g. gel electrophoresis or/and chromatography;
(c) the short RNA fraction is used to probe a microarray chip that comprises nucleic acid fragments of the RNA target, preferably arrays of synthetic oligonucleotides;
(d) the data from the microarray analysis are then interpreted to deduce the parts of the target molecule are effectively single-stranded; and optionally
(e) a model is built for the secondary or tertiary structure of the target RNA molecule.

It is also possible to adapt the above algorithm for studying RNA-protein interactions. Binding of a protein to RNA target has to decrease accessibility of the binding site and can as well alter RNA secondary and tertiary structure. Therefore, information on the RNA-protein interaction can be gleaned from comparing two data sets: one for the individual RNA target, the other one for the mixture of the RNA target and an RNA-binding protein. In its currently preferred form, the method for studying RNA-protein interaction is as follows:
(a) providing RNA molecule and RNA-binding protein;
(b) contacting said RNA and a solution of RNA-binding protein under conditions sufficient for the RNA-protein interaction so that to form an experimental mixture, and in a separate vessel, contacting said RNA with a control solution that lacks said RNA-binding protein so that to form a control mixture.
(c) contacting said experimental and control mixtures with the protein of this invention under conditions sufficient for RNA synthesis;
(d) recovering and optionally purifying the newly produced labeled nucleic acid species from both reaction mixtures;
(e) using the two sets of labeled nucleic acid species as probes for two identical microarray chips that comprise nucleic acid fragments of the RNA target;
(f) interpreting data from the two microarray analyses to deduce which parts of the target molecule are accessible for the short RNA synthesis;
(g) comparing the two data sets to determine the difference between RNA in experimental and control mixtures; and optionally
(h) interpreting the difference between the two data sets as a model for RNA-protein interaction

The novel methods for studying RNA structure and RNA-protein interaction can be applied to very long RNA targets, such as ssRNAs longer than 1 kb. This is an obvious advantage over the previously described techniques involving chemical or enzymatic probing of RNA molecules, which produce information for small RNA fragments limited to several hundreds of nucleotides.

Although in the currently preferred embodiment both above methods are practiced with ssRNA targets and RNA binding proteins, in other embodiments ssDNA targets and ssDNA binding proteins can be employed to obtain information on ssDNA spatial structure and DNA-protein interactions, respectively.

Further aspects and advantages of this invention will become apparent from the appended examples and claims.

### EXAMPLES

### Example 1

### Expression and Purification of Recombinant Soluble QDE-1 and Its Genetic Derivatives

Sequence Analysis of Cellular RdRP-Like Proteins

Amino acid sequences of tomato RdRP and cellular RdRP-like proteins genetically shown to be involved in PTGS were aligned using the ClustalW algorithm (Thompson et al., 1994). The similarity plot built up on the alignment data demonstrates that the amino termini of these proteins are noticeably more divergent (<20% similarity), than the carboxyterminal parts (FIG. 1B). Within this conserved region, one particular span shows the highest similarity (“HS” in FIG. 1B; and FIG. 1C). If RdRP-like proteins indeed possess RNA-polymerizing activity, the elements crucial for this function are likely to reside within the C-terminal domain. In viral RdRPs, two conserved carboxylates located within motifs A and C catalyze the nucleotidyl transfer (Butcher et al., 2001; Hansen et al., 1997; Steitz, 1998). Based on the sequence context, the third aspartate from the GSDLDGDX (SEQ ID NO: 13) (X can be any amino acid) block in the HS sequence may correspond to the catalytic aspartate from the C motif of viral RdRPs (GDD/N; (Poch et al., 1989).

Construction of Expression Plasmids

To construct S. cerevisiae expression plasmid encoding His-tagged full-length QDE-1 (pEM41), wild-type intronless QDE-1 gene was PCR-amplified from N. crassa genomic DNA using a mixture of Turbo Pfu (Stratagene) and Taq (Promega) DNA polymerases and the primers 5′-GCCAAGCTTCCATGAACCCTATTACTCCTA-3′ (SEQ ID NO:7) (qde1_up3) and 5′-CCGAATTCATAATCGCCATTCCCTGTGA-3 (SEQ ID NO: 8)(qde1_down3). The PCR fragment digested with HindIII and EcoRI was gel-purified and ligated with the similarly cut vector pYES2/CT (Invitrogen). Sequence of the QDE-1 insert was sequenced in several pEM41 clones. One clone that did not differ from the previously published sequence on the amino acid level was selected for further experiments.

Two plasmids encoding ΔN (QDE-1 missing 1-376 aa) were constructed, pEM42 and pEM46. For this purpose, the 3′-terminal part of QDE-1 gene was amplified from pEM41 using Turbo Pfu DNA polymerase and the primers 5′-GCTCAAATCCCATGGCTCGGAGCGAAGAAA-3′ (SEQ ID NO: 9) (qde1_up2) and 5′-CCGAATTCTAATAATCGCCATTCCCTGTGA-3′ (SEQ ID NO: 10) (qde1_down1). The PCR fragment was treated with NcoI-EcoRI and ligated with the similarly cut vector pET21d (Novagen) to obtain an E. coli expression plasmid pEM42. The NcoI-Eco81I fragment of QDE-1 was excised from pEM42 and inserted into pEM41 to substitute the QDE-1 HindIII-Eco81I fragment, the NcoI and HindIII cut termini being filled in with the Klenow fragment of DNA polymerase I. The resultant plasmid pEM46 was used to produce His-tagged QDE-1ΔN in S. cerevisiae.

Expression and Purification

The expression plasmid pEM41 was introduced into S. cerevisiae INVSc1 (Invitrogen, his3Δ1/his3Δ1 leu2/leu2 trp1-289/trp1-289 ura3-52/ura3-52) and QDE-1 expression was induced with galactose. As judged by the Coomassie-stained SDS-PAGE, the cells produced a ˜163 kDa protein that was missing in the non-induced INVSc1(pEM41) and induced INVSc1(pYES2/CT) controls. His-tag specific antibodies recognized the protein on Western blots, further confirming its identity (data not shown). In the initial purification experiments, induced INVSc1(pEM41) cells were disrupted in a buffer containing 50 mM Tris-HCl, pH 8.0, 300 mM NaCl in the presence of phenylmethylsulphonylfluoride (PMSF). Under these conditions, most of the 163 kDa protein was in an insoluble form. The supernatant fraction contained minute amounts of His-tagged species, mostly of lower molecular weights (not shown). We therefore optimized cell-disruption buffer by increasing the NaCl concentration in the cell disruption buffer to 1 M and introducing nonionic detergents. In addition, the pH was increased to 9.3 to depart from the theoretical isoelectric point of the His-tagged QDE-1 (pI˜8.4), and a protease inhibitor cocktail was used to block proteolysis. Under these conditions, approximately one third of the full-length QDE-1 could be recovered in a pure soluble form by one-step purification on a nickel-chelating column (FIG. 2A). A similar expression and purification strategy was utilized to obtain soluble truncated QDE-1 missing 376 aa from the N terminus (ΔN), as well as ΔN with the D1011A point mutation (ΔN^{DA}; numeration for the full length QDE-1) destroying the potential catalytic aspartate (FIG. 2A; and see FIG. 1C). Typical yields of purified proteins were ˜0.5 mg (QDE-1) or 3-5 mg (ΔN and ΔN^{DA}) per 1 L of yeast culture. The currently preferred expression and purification protocol is as follows:

S. cerevisiae cultures (40 ml) grown overnight at 30° C. in the SC^{-Ura,+Glu} minimal medium at 240 rpm until OD₆₀₀ ˜3. The cells were collected by centrifugation for 5 min at 1500 g, room temperature, washed once with 20 ml SC^{-Ura,+Raf} and resuspended in 300 ml of SC^{-Ura,+Gal,+Raf} to a final OD₆₀₀ of 0.4. The shaking was continued at 28° C. for 22 h. The cells were then harvested by centrifugation for 5 min at 5000 g, 4° C., washed with 100 ml of ice-cold water and resuspended in 15 ml of ice-cold buffer H-5 (50 mM Tris-HCl, pH 9.3; 1M NaCl; 1% Triton X-100; 1% Tween 20; 5% glycerol; 5 mM imidazole) containing Complete Mini EDTA-free protease inhibitor cocktail (Roche; 1 tablet per 7.5 ml). The suspension was stored at −80° C. in 5 ml aliquots until needed. Protein purification was done at 4° C. Thawed cell suspension (5 ml) was passed twice through a precooled French pressure cell at ˜20,000 psi. PMSF was added to 1 mM after the first passage. Alternatively, the cells were disrupted by shaking with acid-washed glass beads. The lysate was centrifuged at 13,000 g for 15 min and the supernatant was loaded onto a 1 ml Ni-NTA column (Qiagen) equilibrated with buffer M-5 (50 mM Tris-HCl, pH 8.9; 300 mM NaCl; 0.5% Triton X-100; 0.5% Tween 20; 5 mM imidazole). The column was washed with 20 ml of M-5 and 10 ml of M-50 (50 mM Tris-HCl, pH 8.9; 300 mM NaCl; 0.5% Triton X-100; 0.5% Tween 20; 50 mM imidazole). Recombinant proteins were eluted from the column with M-200 (50 mM Tris-HCl, pH 8.9; 300 mM NaCl; 0.5% Triton X-100; 0.5% Tween 20; 200 mM imidazole). Fractions were analyzed by SDS-PAGE and the protein concentration was determined by comparing protein bands with bands containing known amounts of bovine serum albumin (BSA). Purified proteins were stored on ice for at least 2 weeks without detectable loss of specific activity. For the negative controls, we used INVSc1 cells containing pYES2/CT and pYES2/CT/lacZ plasmids (Invitrogen).

QDE-1 derived ΔN protein could be also produced in E. coli BL21(DE3) transformed with pEM42 using previously published expression protocol (Makeyev and Bamford, 2000). However, the recombinant protein was in an insoluble form despite our optimizations (not shown).

### Example 2

### Characterization of RNA-Polymerization Activity of QDE-1 and Its Derivatives

RNA Templates

Synthetic ssRNA templte for RdRP assays were prepared by in vitro run-off transcription with T7 RNA polymerase in principle as described (Gurevich et al., 1991; Makeyev et al., 1996). References for the plasmids used for this purpose are given in figure legends. Plasmid pEM54 was derived from pTZluc(-stop) (Makeyev et al., 1996) by deleting the HindIII-EcoRV 5′-terminal fragment of the luciferase gene. Viral RNAs were extracted from purified virus particles (φ6, LA, and TMV) with phenol and chloroform, precipitated with ethanol and dissolved in water or 10 mM Tris-HCl, pH 8.0, 0.1 mM EDTA. RNA concentration was measured by optical density at 260 nm and the quality was determined by electrophoresis in standard or/and formaldehyde-containing agarose gels (Sambrook and Russell, 2001).

Purified QDE-1 is Enzymatically Active in vitro

The isolated QDE-1 was assayed for its possible RdRP activity in 10 μl reaction mixtures containing 50 mM HEPES-KOH, pH 7.8, 20 mM ammonium acetate (NH₄OAc), 6% (w/v) PEG4000, 5 mM MgCl₂, 0.1 MM EDTA, 0.1% Triton X-100, 1 mM each of ATP and GTP, 0.2 mM each of CTP and UTP, 0.8 unit/μl RNasin, and 0.1 mCi/ml of [α-³²P]UTP (˜3000 Ci/mmol; Amersham Biosciences). 90 μg/ml firefly luciferase (luc) mRNA was used as a template. Reactions were initiated by the addition of polymerase preparation or buffer M-200 followed by incubation at 30° C. for 1 h. Reaction products were analyzed by standard agarose gel electrophoreses followed by autoradiography. The presence of QDE-1 in the mixture correlated with the appearance of two distinct reaction products (FIG. 2B, lanes 1 and 2), one migrating in native agarose gel slightly slower than the ssRNA template (lower band), the other one co-migrating with the dsRNA of the template length (upper band). To rule out that the products were synthesized by yeast enzymes contaminating the QDE-1 preparation, we assayed similarly purified control fractions from galactose-induced cells containing vector (pYES2/CT) or plasmid encoding His-tagged LacZ (pYES2/CT/lacZ; Invitrogen). No polymerization products were detected in either of the two cases (not shown).

Observed Activity is a Function of QDE-1 C-Terminal Domain

To characterize the newly found activity, purified soluble ΔN (carboxyterminal fragment of QDE-1) was assayed with the luc template as described above. The two labeled RNA products were apparent on the autoradiogram, similarly to the QDE-1 catalyzed reaction (FIG. 2B, lane 3). Specific activity of ΔN is similar to that of QDE-1, thus implicating the C-terminal domain in the catalysis.

Notably, no activity was detected when ΔN^{DA} (ΔN with the putative catalytic residue Asp1011 changed to Ala) was assayed under the same conditions (FIG. 2B, lanes 4 and 5). To make sure that the loss of activity in ΔN^{DA} is not because of an alteration in the protein fold, both ΔN and ΔN^{DA} were subjected to gel-filtration under native conditions, as depicted in FIG. 2C for ΔN. There was no detectable difference in the position or shape of the ΔN and ΔN^{DA} peaks, thus indicating that ΔN^{DA} is properly folded (not shown). Position of the ΔN protein peak coincided with the peak of enzymatic activity, thus providing additional evidence against possible contamination with cellular enzymes (FIG. 2D).

QDE-1 Catalyzes RNA-Dependent RNA Polymerization

To ascertain that the reaction catalyzed by QDE-1 was RNA-dependent RNA polymerization, the enzyme was assayed in the mixtures lacking either template or unlabeled nucleotides (ATP, CTP and GTP). As expected, no labeled products were formed in these two reactions (FIG. 3A, lanes 2 and 4). Purified RdRP subunit of dsRNA virus φ6 (φ6Pol), used here as a control, produced full-length dsRNA product, which was expected from our previous work (FIG. 3A, lane 1; and see (Makeyev and Bamford, 2000). Similarly to φ6Pol, the upper band in the QDE-1 directed reactions is likely to arise through the end-to-end polymerization. Because of its intermediate mobility between the ss and ds species, the faster migrating products can appear as a result of incomplete synthesis, with only part of the template being converted into the double-stranded form.

To examine whether the nucleotide composition of the RNA products was instructed by template, we took advantage of the “nearest neighbor” analysis that allows one to determine a distribution of the nucleosides 5′-adjacent to α-labeled nucleoside-5′-monophosphates incorporated in the RNA product. QDE-1 was incubated with either luc RNA or poly(A) homopolymer in the presence of 0.2 mM each of the four unlabeled NTPs and [α³²P]UTP. In both reactions, labeled products were readily detectable by agarose gel electrophoresis and TCA precipitation (not shown). The reaction products were digested with RNase T2 to generate nucleoside-3′-monophosphates (Ap, Cp, Gp and Up), which were separated by thin-layer chromatography (TLC). In the case of template-dependent synthesis, all four nucleoside-3′-monophosphates will carry labeled phosphate for the luc template, with the Cp, Gp, Ap and Up distributed as 1.0 to 1.1 to 1.4 to 1.8. Only labeld Up is epected for the poly(A)-programmed reaction. FIG. 3B confirms these predictions completely.

QDE-1 Can Initiate RNA Synthesis de novo

Many RdRPs utilize primer-independent initiation mechanism (Butcher et al., 2001; Laurila et al., 2002)and references therein). To test whether QDE-1 can also support primer-independent (de novo) initiation, the polymerase was assayed in the presence of γ-labeled nucleotides. The first 5′-terminal nucleotide of daughter strand initiated de novo should retain its triphosphate moiety, whereas only α-phosphates will be incorporated in the case of primed synthesis. Labeled RNA products were produced both in the presence of [γ-³²P]GTP and [γ-³²P]ATP, although the incorporation efficiency of [γ-³²P]GTP was noticeably higher. FIG. 3C shows that using the 5′Δmₛ⁺ RNA template (a φ6-specific RNA with the . . . UUCC-3′ terminus; (Makeyev and Bamford, 2000) QDE-1 incorporated the label predominantly into the lower band (partially double-stranded species), while the upper band (full-length dsRNA) was labeled very weakly. A similar pattern was observed for the luc template (not shown). This indicates that the partial dsRNA species might contain multiple copies of de novo initiated daughter strands annealed to the template.

To exclude that the γ-phosphate was introduced through a γ-phosphate transfer rather than RNA polymerization, we treated labeled RNAs with the guanosyl-specific RNase T1 and analyzed the digest by TLC (Vasiljeva et al., 2000). The label was retrieved from the RNA products of QDE-1 in the form of pppGp. In the control digestion, containing the oligonucleotide 5′-GUUUUCACCCUAUCCUCCCC-3′ (SEQ ID NO: 11) labeled at the 5′ α-position with T4 polynucleotide kinase, the label was released in the form of pGp, as expected (FIG. 3D).

Effect of Divalent Cations

Since all known RdRPs are sensitive to divalent metal ions, we studied the effect of Mg²⁺, Mn²⁺, and Ca²⁺ on the QDE-1-catalyzed reaction (FIG. 3E). An increase in Mg²⁺ concentration did not affect the band intensity within the 5-9 mM range (lanes 1-4). Mn²⁺ stimulated the synthesis of both reaction products at 1 mM concentration (lane 5). However further increase in [Mn²⁺] to 2-4 mM led to the disappearance of the upper band, with only little effect on the lower one (lanes 6-7). Calcium ions inhibited the RdRP reaction almost completely at 4 mM (lane 10). At lower Ca²⁺ concentrations, the synthesis of the faster migrating product was affected more severely than the upper one (lanes 8-9). The effects of Mn²⁺ and Ca²⁺ on the distribution of the RNA products are consistent with the idea that QDE-1 may utilize two polymerization modes with different reaction optima.

QDE-1 Accepts a Number of ssRNA Templates

To assess template preferences of QDE-1, we also assayed QDE-1 with several other single-stranded templates, such as green fluorescent protein (GFP) mRNA, genomic RNA of tobacco mosaic virus (TMV), as well as several N. crassa and φ6-specific RNAs (FIG. 4A and FIG. 6A). In all cases, QDE-1 produced full-length dsRNA and the partially double-stranded species, exactly as for the luc template. In some experiments, when reaction products were purified before electrophoresis by gel-filtration on Sephadex G-50 equilibrated with water, bands migrating as ˜20 nt ssRNA were also apparent on the autoradiograms (arrowhead in FIG. 4A). Neither these, nor the partially dsRNA products were present in the corresponding φ6Pol lanes, thus suggesting that they might be a specific trait of the cellular RdRP involved in the RNA silencing process.

The Nature of the Reaction Products

To identify the nature of the QDE-1 polymerization products, two independent approaches were employed. First, RdRP products were separated by electrophoresis in a formaldehyde-containing gel (FIG. 4B). Under these conditions, the RNA strands of dsRNA products are completely denatured and migrate according to their length. Three bands were detected for the luc mRNA-programmed reaction: a faint band of the template length (1×), a more intense band twice as long as the template (2×), and the strongest band migrating at the ˜20 nt position (sRNA, for brevity). φ6Pol only produced the 1× and 2× products, with no signs of the sRNA. It has been shown earlier that the 1× species arises as a result of de novo initiated RNA synthesis, whereas the 2× originates by extending the folded back 3′ end of the template (so-called “back-priming”; Laurila et al., 2002). Thus, QDE-1 used predominantly the back-priming mode to produce the complete or nearly complete copy of the luc mRNA. On the native gel, this product migrates as the template-length dsRNA. (see FIG. 4A). The sRNA products are apparently derived from the incomplete dsRNA product. Since this product migrates noticeable slower than the luc mRNA on the native gel, multiple copies of complementary sRNA are likely produced on each template molecule. It is obvious from FIG. 4B that QDE-1 employs predominantly de novo initiation mechanism to produce small amounts of nearly full-length dsRNA on the GFP and TMV templates. And again, complementary sRNAs migrating in the 20 nt region represent the major reaction product.

For the second approach, we used the property of RNase I to degrade ssRNA but not dsRNA at a high ionic strength. If QDE-1 produces two discrete types of dsRNA elements, as anticipated from the above experiments, two products will be protected from the RNase I digestion: (i) short dsRNAs and (ii) (nearly) full-length dsRNA (FIG. 4C). To perform the RNase protection assay, QDE-1 reaction mixtures, containing labeled luc RNA, the four unlabeled NTPs and no labeled nucleotides, were quenched by adding 250 mM NH₄OAc, 10 mM EDTA. The mixtures were then supplemented with 0.05 unit/μl of RNase I (RNase ONE; Promega) or an equal volume of RNAse ONE 1×buffer, and incubated for 1 h at 30° C. The reactions were stopped by the addition of 0.2% SDS and the products were analyzed by standard agarose gel electrophoresis. Both RNase-resistant species predicted in FIG. 4C were indeed detected after the electrophoretic separation (FIG. 4D), thus supporting our conclusions.

sRNAs are 9-21 Nucleotide Long

To accurately determine the lengths of the sRNAs, we carried out polymerization reactions with different RNA template in the presence of γ-labeled GTP. After the incubation at 30° C., reaction products were analyzed using a high-resolution urea-containing PAGE. Regardless of the template, sRNAs appeared as a population of 9-21-mer oligonucleotides with occasional week bands of shorter and longer products (FIG. 5 and FIG. 6B). The sRNA patterns by QDE-1 and ΔN were identical; no sRNA was detected in the φ6Pol controls (FIG. 5). Similar patterns were obtained when α-labeled UTP was used instead of γ-label, with the only difference that the relative intensity of longer products was higher (not shown).

Reactions with Primed ssRNA and Blunt-Ended dsRNA Templates

Models 1 and 2 in FIG. 1A imply that QDE-1 can utilize dsRNA templates and/or extend complementary primers annealed to a ssRNA template. To address these predictions, we assayed QDE-1 and QDE-1ΔN with blunt-ended dsRNAs extracted from φ6 virions or yeast LA virus-like particles (FIG. 10A). No labeled products were detected even after prolonged exposures. In contrast, φ6Pol produced readily detectable dsRNA labeled products that were synthesized via a semi-conservative (strand-displacement) mechanism, as expected (Makeyev and Bamford, 2000).

To assess the primer extension capacity of QDE-1, synthetic RNA oligonucleotide 5′-CGACUCAUGGACCUUGGGAG-3′ (SEQ ID NO: 12) was labeled with T4-PNK and [γ-³²P]ATP, annealed with sR5 RNA template (T7 transcript of pLM659 cut with EcoRV; Gottlieb et al., 1992) and assayed in the RdRP reaction mixtures (see description above) containing 40 μg/ml of QDE-1 (or φ6Pol) and no labeled nucleotides. As a control, the same primer-template substrate was incubated for 1 h at 37° C. in 10 μl mixtures containing 5 units of AMV-RT (Sigma), 8 units of RNasin, and 0.5 mM each of the four deoxynucleotide triphosphates in the recommended buffer. The reaction products were separated by 6% PAGE containing 7.5 M urea (FIG. 10B).

A detectable amount of the full-length extended product was detected in the QDE-1 lane, with no band at this position in the “buffer only” control. A similar product was also visible in the φ6Pol lane. Under the conditions employed, reverse transcriptase of avian myeloblastosis virus (AMV-RT) produced 10-20 times more of the extended product (cDNA), than either of the two RdRPs (FIG. 10C).

### Example 3

### Downstream Applications of QDE-1 Polymerase and its Derivatives

Using sRNAs as Sequence-Specific Probes

If sRNA are distributed evenly along the entire template, they can be purified from their encoding templates and other components of RdRP mixtures and used as probes in molecular and cellular techniques that are based on nucleic acid hybridization. For this purpose, γ-³²P labeled sRNAs synthesized on the luc RNA were used as probes for Northern blotting (FIG. 9A). Six RNAs were used as the hybridization targets: four sense fragments of luc RNA spanning different regions as shown in FIG. 9B, full-length antisense luc RNA (a-luc) and a control sR5 RNA originating from the φ6 s⁺ RNA and containing no homology to the luciferase gene.

To prepare the sRNA probe for Northern blotting, luciferase mRNA was incubated with QDE-1 in the presence of the four unlabeled NTP and [γ-³²P]GTP as outlined above. RNA products were denatured and separated using gel-electrophoresis in a low melting point agarose gel. The zone containing labeled sRNAs was excised from the gel; the sRNAs were recovered by melting the agarose at 70° C. and used for probing target RNAs without further purification. Target RNAs (specified above) were separated in formaldehyde-containing gels and transferred to Hybond-N+ (Amersham Biosciences) as described (Sambrook and Russell, 2001). The membranes were blocked in 6×SSC, 7% SDS for 4 h at 68° C., which was followed by overnight hybridization at 42° C. in the same buffer containing the sRNA probe (˜10⁵ cpm/ml). After hybridization, the membranes were washed three times with 2×SSC at room temperature followed by 2 washes with 2×SSC, 0.1% SDS at 42° C. (30 min each wash). The membranes were air-dried and analyzed with a phosphoimager.

The result of this experiment is presented in FIG. 9C. All four sense RNAs were recognized by the probe, thus suggesting that the sRNA population contains species complementary to the different template segments. As expected, no signal was detected in the sR5 lane. There was a very weak labeling of the a-luc RNA band, which could be formally explained by the presence of low amounts of sense sRNAs in the probe. However, this might also be an artifact of hybridization, since low stringency conditions were used.

Incorporation of Chemically Modified Nucleotides into Newly Produced RNA

It is advantageous for many applications to generate RNA products containing non-radioactive labels. To explore this possibility for QDE-1 and its derivatives, reactions were carried out with biotin-11-CTP where the label is tethered to the nucleotide base. Reaction products were immobilized on a membrane and probed with HRP-streptavidin conjugate as outlined in FIG. 12A. A strong signal was detected for the mixtures containing both polymerase (ΔN) and the template (luc), thus indicating that the labeled cytosine can indeed be incorporated into the product fraction. A similar result was obtained when coumarin-5-CTP was used as a label and the coumarin fluorescence was measured in the purified reaction product fraction (FIG. 12B).

RNA Products of QDE-1 Induce RNAi in C. elegans

To address the biological activity of the QDE-1 reaction products, we took advantage of the RNAi approach. Of the organisms both amenable to RNAi and also having an RdRP component of the PTGS pathway, C. elegans is the most convenient experimental model. Basic C. elegans techniques were as described (Lewis and Fleming, 1995). For the RNAi experiment, 20 μg GFP mRNA was incubated with 1 μg QDE-1 or an equal volume of buffer M-200 in 50 μl reaction mixtures for 1 h at 30° C., as described in Example 2. Reaction products were extracted with phenol-chloroform and precipitated with ethanol in the presence of 2.5 M ammonium acetate. The pellets were dissolved in 15 μl of M9 buffer (Brenner, 1974) additionally supplemented with 3 mM spermidine and 0.5 mg/ml BSA. 5 μl aliquots of the RNA solutions were mixed with 0.5 μl of 20 mM CaCl₂ in 0.5 ml eppendorf tubes immediately prior to use. 5-10 young adult hermaphrodites (C. elegans AZ218; pharyngeal GFP expression; Praitis et al., 2001) were washed with M9 and soaked in each tube for 24 h at 20° C. The worms were then placed onto NGM plates sparsely seeded with E. coli OP50-1 for another 24 h at 20° C. The worms were further transferred to fresh NGM plates painted with a grid of OP50-1 for another 72 h (20° C.). F1 progeny from these plates was scored for the expression of GFP in the pharynx using an Olimpus IX70 microscope. GFP expression was detectably silenced in ˜60% of the F1 generation from the parents treated with the QDE-1 reactions products, whereas all F1 worms showed detectable pharyngeal GFP fluorescence in the M-200 buffer control.

### LITERATURE CITED

Patents

U.S. Pat. No. 6,218,142
   Journal Articles
Barstead, R. (2001). Genome-wide RNAi. Curr. Opin. Chem. Biol. 5, 63-6.
Baulcombe, D. (2002). RNA silencing. Curr. Biol. 12, R82-4.
Bell-Pedersen, D., Dunlap, J. C., and Loros, J. J. (1992). The Neurospora circadian clock-controlled gene, ccg-2, is allelic to eas and encodes a fungal hydrophobin required for formation of the conidial rodlet layer. Genes Dev. 6, 2382-94.
Bernstein, E., Caudy, A. A., Hammond, S. M., and Hannon, G. J. (2001). Role for a bidentate ribonuclease in the initiation step of RNA interference. Nature 409, 363-6.
Bowman, B. J., Allen, R., Wechser, M. A., and Bowman, E. J. (1988). Isolation of genes encoding the Neurospora vacuolar ATPase. Analysis of vma-2 encoding the 57-kDa polypeptide and comparison to vma-1. J. Biol. Chem. 263, 14002-7.
Bowman, E. J., Tenney, K., and Bowman, B. J. (1988). Isolation of genes encoding the Neurospora vacuolar ATPase. Analysis of vma-1 encoding the 67-kDa subunit reveals homology to other ATPases. J. Biol. Chem. 263, 13994-4001.
Brenner, S. (1974). The genetics of Caenorhabditis elegans. Genetics 77, 71-94.
Butcher, S. J., Grimes, J. M., Makeyev, E. V., Bamford, D. H., and Stuart, D. I. (2001). A mechanism for initiating RNA-dependent RNA polymerization. Nature 410, 235-40.
Catalanotto, C., Azzalin, G., Macino, G., and Cogoni, C. (2002). Involvement of small RNAs and role of the qde genes in the gene silencing pathway in Neurospora. Genes Dev. 16, 790-5.
Chalfie, M., Tu, Y., Euskirchen, G., Ward, W. W., and Prasher, D. C. (1994). Green fluorescent protein as a marker for gene expression. Science 263, 802-5.
Chicas, A., and Macino, G. (2001). Characteristics of post-transcriptional gene silencing. EMBO Rep. 2, 992-6.
Cogoni, C., and Macino, G. (1999). Gene silencing in Neurospora crassa requires a protein homologous to RNA-dependent RNA polymerase. Nature 399, 166-9.
Cogoni, C., and Macino, G. (1999). Homology-dependent gene silencing in plants and fungi: a number of variations on the same theme. Curr. Opin. Microbiol 2, 657-62.
Cogoni, C., and Macino, G. (1997). Isolation of quelling-defective (qde) mutants impaired in posttranscriptional transgene-induced gene silencing in Neurospora crassa. Proc. Natl. Acad. Sci. U.S.A. 94, 10233-8.
Cogoni, C., and Macino, G. (2000). Post-transcriptional gene silencing across kingdoms. Curr. Opin. Genet. Dev. 10, 638-43.
Cogoni, C., and Macino, G. (1999). Posttranscriptional gene silencing in Neurospora by a RecQ DNA helicase. Science 286, 2342-4.
Dalmay, T., Hamilton, A., Rudd, S., Angell, S., and Baulcombe, D. C. (2000). An RNA-dependent RNA polymerase gene in Arabidopsis is required for posttranscriptional gene silencing mediated by a transgene but not by a virus. Cell 101, 543-53.
Elbashir, S. M., Lendeckel, W., and Tuschl, T. (2001). RNA interference is mediated by 21- and 22-nucleotide RNAs. Genes Dev. 15, 188-200.
Fagard, M., Boutet, S., Morel, J. B., Bellini, C., and Vaucheret, IL (2000). AGO1, QDE-2, and RDE-1 are related proteins required for post-transcriptional gene silencing in plants, quelling in fungi, and RNA interference in animals. Proc. Natl. Acad. Sci. U.S.A. 97, 11650-4.
Fire, A. (1999). RNA-triggered gene silencing. Trends Genet. 15, 358-63.
Fire, A., Xu, S., Montgomery, M. K., Kostas, S. A., Driver, S. E., and Mello, C. C. (1998). Potent and specific genetic interference by double-stranded RNA in Caenorhabditis elegans. Nature 391, 806-11.
Fjose, A., Ellingsen, S., Wargelius, A., and Seo, H. C. (2001). RNA interference: mechanisms and applications. Biotechnol Annu. Rev. 7,31-57.
Fraser, A. G., Kamath, R. S., Zipperlen, P., Martinez-Campos, M., Sohrmann, M., and Ahringer, J. (2000). Functional genomic analysis of C. elegans chromosome I by systematic RNA interference. Nature 408, 325-30.
Gitlin, L., Karelsky, S., and Andino, R. (2002). Short interfering RNA confers intracellular antiviral immunity in human cells. Nature 418, 430-4.
Gonczy, P., Echeverri, C., Oegema, K., Coulson, A., Jones, S. J., Copley, R. R., Duperon, J., Oegema, J., Brehm, M., Cassin, E., Hannak, E., Kirkham, M., Pichler, S., Flohrs, K., Goessen, A., Leidel, S., Alleaume, A. M., Martin, C., Ozlu, N., Bork, P., and Hyman, A. A. (2000). Functional genomic analysis of cell division in C. elegans using RNAi of genes on chromosome III. Nature 408, 331-6.
Gottlieb, P., Strassman, J., Qiao, X., Frilander, M., Frucht, A., and Mindich, L. (1992). In vitro packaging and replication of individual genomic segments of bacteriophage phi 6 RNA. J. Virol. 66, 2611-16.
Gurevich, V. V., Pokrovskaya, I. D., Obukhova, T. A., and Zozulya, S. A. (1991). Preparative in vitro mRNA synthesis using SP6 and T7 RNA polymerases. Anal. Biochem. 195, 207-13.
Hamilton, A. J., and Baulcombe, D. C. (1999). A species of small antisense RNA in posttranscriptional gene silencing in plants. Science 286, 950-2.
Hammond, S. M., Bernstein, E., Beach, D., and Hannon, G. J. (2000). An RNA-directed nuclease mediates post-transcriptional gene silencing in Drosophila cells. Nature 404, 293-6.
Hanna, M., and Szostak, J. W. (1994). Suppression of mutations in the core of the Tetrahymena ribozyme by spermidine, ethanol and by substrate stabilization. Nucleic Acids Res. 22, 5326-31.
Hansen, J. L., Long, A. M., and Schultz, S. C. (1997). Structure of the RNA-dependent RNA polymerase of poliovirus. Structure 5, 1109-22.
Hutvagner, G., and Zamore, P. D. (2002). RNAi: nature abhors a double-strand. Curr. Opin. Genet. Dev. 12, 225-32.
Jacque, J. M., Triques, K., and Stevenson, M. (2002). Modulation of HIV-1 replication by RNA interference. Nature 418, 435-8.
Ketting, R. F., Haverkamp, T. H., van Luenen, IL G., and Plasterk, R. H. (1999). Mut-7 of C. elegans, required for transposon silencing and RNA interference, is a homolog of Werner syndrome helicase and RNaseD. Cell 99, 133-41.
Kolb, V. A., Makeyev, E. V., and Spirin, A. S. (2000). Co-tanslational folding of an eukaryotic multidomain protein in a prokaryotic translation system. J. Biol. Chem. 275, 16597-601.
Laurila, M. R., Makeyev, E. V., and Bamford, D. H. (2002). Bacteriophage phi6 RNA-dependent RNA polymerase: Molecular details of initiating nucleic acid synthesis without primer. J. Biol. Chem. in press.
Lewis, J. A., and Fleming, J. T. (1995). Basic culture methods. Methods Cell Biol. 48, 3-29.
Lipardi, C., Wei, Q., and Paterson, B. M. (2001). RNAi as random degradative PCR: siRNA primers convert mRNA into dsRNAs that are degraded to generate new siRNAs. Cell 107, 297-307.
Maeda, I., Kohara, Y., Yamamoto, M., and Sugimoto, A. (2001). Large-scale analysis of gene function in Caenorhabditis elegans by high-throughput RNAi. Curr. Biol. 11, 171-6.
Makeyev, E. V., and Bamford, D. H. (2000). The polymerase subunit of a dsRNA virus plays a central role in the regulation of viral RNA metabolism. EMBO J. 19, 6275-84.
Makeyev, E. V., and Bamford, D. H. (2000). Replicase activity of purified recombinant protein P2 of double-stranded RNA bacteriophage phi6. EMBO J. 19, 124-33.
Makeyev, E. V., Kolb, V. A., and Spirin, A. S. (1996). Enzymatic activity of the ribosome-bound nascent polypeptide. FEBS Lett. 378, 166-70.
Martens, H., Novotny, J., Oberstrass, J., Steck, T. L., Postlethwait, P., and Nellen, W. (2002). RNAi in Dictyostelium: the role of RNA-directed RNA polymerases and double-stranded RNase. Mol. Biol. Cell 13, 445-53.
Montgomery, M. K., Xu, S., and Fire, A. (1998). RNA as a target of double-stranded RNA-mediated genetic interference in Caenorhabditis elegans. Proc. Natl. Acad. Sci. U.S.A. 95, 15502-7.
Mourrain, P., Beclin, C., Elmayan, T., Feuerbach, P., Godon, C., Morel, J. B., Jouette, D., Lacombe, A. M., Nikic, S., Picault, N., Remoue, K., Sanial, M., Vo, T. A., and Vaucheret, H. (2000). Arabidopsis SGS2 and SGS3 genes are required for posttranscriptional gene silencing and natural virus resistance. Cell 101, 533-42.
Nishikura, K. (2001). A short primer on RNAi: RNA-directed RNA polymerase acts as a key catalyst. Cell 107, 415-8.
Novina, C. D., Murray, M. F., Dykxhoorn, D. M., Beresford, P. J., Riess, J., Lee, S. K., Collman, R. G., Lieberman, J., Shankar, P., and Sharp, P. A. (2002). siRNA-directed inhibition of HIV-1 infection. Nat. Med. 8, 681-6.
Plasterk, R. IL A. (2002). RNA Silencing: The Genome's Immune System. Science 296, 1263-1265.
Poch, O., Sauvaget, I., Delarue, M., and Tordo, N. (1989). Identification of four conserved motifs among the RNA-dependent polymerase encoding elements. EMBO J. 8, 3867-74.
Praitis, V., Casey, E., Collar, D., and Austin, J. (2001). Creation of low-copy integrated transgenic lines in Caenorhabditis elegans. Genetics 157, 1217-26.
Quigley, G. J., Teeter, M. M., and Rich, A. (1978). Structural analysis of spermine and magnesium ion binding to yeast phenylalanine transfer RNA. Proc. Natl. Acad. Sci. U.S.A. 75, 64-8.
Saitou, N., and Nei, M. (1987). The neighbor-joining method: a new method for reconstructing phylogenetic trees. Mol. Biol. Evol. 4, 406-25.
Sambrook, J., and Russell, D. (2001). Molecular cloning: a laboratory manual (3rd edition) (Cold Spring Harbor, N.Y.: Cold Spring Harbor Laboratory Press).
Schiebel, W., Haas, B., Marinkovic, S., Klanner, A., and Sanger, IL L. (1993). RNA-directed RNA polymerase from tomato leaves. I. Purification and physical properties. J. Biol. Chem. 268, 11851-7.
Schiebel, W., Haas, B., Marinkovic, S., Klanner, A., and Sanger, H. L. (1993). RNA-directed RNA polymerase from tomato leaves. II. Catalytic in vitro properties. J. Biol. Chem. 268, 11858-67.
Schiebel, W., Pelissier, T., Riedel, L., Thalmeir, S., Schiebel, R., Kempe, D., Lottspeich, F., Sanger, H. L., and Wassenegger, M. (1998). Isolation of an RNA-directed RNA polymerase-specific cDNA clone from tomato. Plant Cell 10, 2087-101.
Shiu, P. K., Raju, N. B., Zickler, D., and Metzenberg, R. L. (2001). Meiotic silencing by unpaired DNA. Cell 107, 905-16.
Sijen, T., Fleenor, J., Simmer, P., Thijssen, K. L., Parrish, S., Timmons, L., Plasterk, R. H., and Fire, A. (2001). On the role of RNA amplification in dsRNA-triggered gene silencing. Cell 107, 465-76.
Smardon, A., Spoerke, J. M., Stacey, S. C., Klein, M. E., Mackin, N., and Maine, E. M. (2000). EGO-1 is related to RNA-directed RNA polymerase and functions in germ-line development and RNA interference in C. elegans. Curr. Biol. 10, 169-78.
Steitz, T. A. (1998). A mechanism for all polymerases. Nature 391, 231-2.
Tabara, H., Sarkissian, M., Kelly, W. G., Fleenor, J., Grishok, A., Timmons, L., Fire, A., and Mello, C. C. (1999). The rde-1 gene, RNA interference, and transposon silencing in C. elegans. Cell 99, 123-32.
Thompson, J. D., Higgins, D. G., and Gibson, T. J. (1994). CLUSTAL W: improving the sensitivity of progressive multiple sequence alignment through sequence weighting, position-specific gap penalties and weight matrix choice. Nucleic Acids Res. 22, 4673-80.
Timmons, L., and Fire, A. (1998). Specific interference by ingested dsRNA. Nature 395, 854.
Vasiljeva, L., Merits, A., Auvinen, P., and Kaariainen, L. (2000). Identification of a novel function of the alphavirus capping apparatus. RNA 5′-triphosphatase activity of Nsp2. J. Biol. Chem. 275, 17281-7.
Wassenegger, M., and Pelissier, T. (1998). A model for RNA-mediated gene silencing in higher plants. Plant Mol. Biol. 37, 349-62.
Waterhouse, P. M., Graham, M. W., and Wang, M. B. (1998). Virus resistance and gene silencing in plants can be induced by simultaneous expression of sense and antisense RNA. Proc. Natl. Acad. Sci. U.S.A. 95, 13959-64.
Waterhouse, P. M., Wang, M. B., and Lough, T. (2001). Gene silencing as an adaptive defence against viruses. Nature 411, 834-42.
Yarden, O., Plamann, M., Ebbole, D. J., and Yanofsky, C. (1992). cot-1, a gene required for hyphal elongation in Neurospora crassa, encodes a protein kinase. EMBO J. 11, 2159-66.
Zamore, P. D., Tuschl, T., Sharp, P. A., and Bartel, D. P. (2000). RNAi: double-stranded RNA directs the ATP-dependent cleavage of mRNA at 21 to 23 nucleotide intervals. Cell 101, 25-33.

1-41. (canceled)

42. A method for producing a nucleic acid product, comprising that an isolated polymerase protein is contacted with a nucleic acid template under conditions sufficient for the function of the enzyme, and wherein said polymerase protein is an RNA polymerase capable of producing
(a) short complementary RNA copies of said template, which are scattered throughout the entire template length and, optionally,
(b) template-length complementary RNA copies.

43. The method according to claim 42, wherein said nucleic acid template is DNA or RNA.

44. The method according to claim 42, wherein the ratio of said short and template-length RNA copies can be adjusted by the reaction conditions.

45. The method according to claim 42, wherein the length of said short RNA copies can be adjusted by the reaction conditions.

46. The method according to claim 42, wherein the said short or template-length RNA copies are annealed to the template or denatured from the template.

47. The method according to claim 42, wherein said nucleic acid template is linear or circular.

48. The method according to claim 42, wherein said polymerase originates from a eukaryotic cell.

49. The method according to claim 48, wherein said polymerase originates from an organism selected from the kingdoms of Fungi, Viridiplantae, Metazoa, or the group of Mycetozoa.

50. The method according to claim 48, wherein said polymerase originates from an organism selected from the subset of genera Neurospora, Arabidopsis, Caenorhabditis, and Dictyostelium, preferebly organisms Neurospora crassa, Arabidopsis thaliana, Caenorhabditis elegans, and Dictyostelium discoideum.

51. The method according to claim 50, wherein said polymerase is QDE-1 protein of Neurospora crassa or an altered or a genetically modified derivative of QDE-1.

52. The method according to claim 42, wherein said RNA polymerase is encoded by a nucleic acid sequence selected from the group of:
(a) a nucleic acid sequence comprising the nucleic acid sequence of SEQ ID NO: 1 or SEQ ID NO: 3;
(b) a nucleic acid sequence encoding a polypeptide comprising the amino acid sequence of SEQ ID NO 2 or SEQ ID NO: 4;
(c) a nucleic acid sequence, which differs from the nucleic acid sequence of (a) or (b) due to degeneracy of the genetic code;
(d) a nucleic acid sequence hybridizing to the nucleic acid sequence of (a), (b) and/or (c) under stringent conditions; and
(e) a nucleic acid sequence encoding a polypeptide comprising the amino acids 709 to 1402 of SEQ ID NO:4 or any sequence longer than that up to the sequence comprising the amino acids 2 to 1402 of SEQ ID NO: 2; and
(f) a nucleic acid sequence encoding an amino acid sequence, which shows at least 50% identity to the amino acid sequence of SEQ ID NO: 2.

53. The method according to claim 42, comprising the steps of:
(a) providing ssRNA or ssDNA template;
(b) contacting said ssRNA or ssDNA template in vitro with the polymerase under conditions sufficient for RNA synthesis.

54. The method according to claim 53, wherein said ssRNA template is provided by transcribing a DNA template with a DNA-dependent RNA polymerase, preferably polymerase selected from the group of DNA bacteriophage-encoded DNA-dependent RNA polymerases, most preferably DNA-dependent RNA polymerase of bacteriophage T7, T3 or SP6.

55. The method according to claim 53, wherein the reactions are carried out at the same time or sequentially in the same reaction vessel.

56. The method according to claim 53, wherein the newly produced RNA species are recovered from the reaction mixture.

57. The method according to claim 53, wherein said newly produced RNA strands are annealed with the template to form dsRNA elements or, alternatively, are denatured from the template.

58. The method according to claim 53, wherein RNA synthesis is initiated from the 3′ end of a nucleic acid primer complementary to the RNA or DNA template or RNA synthesis is initiated without a primer.

59. The method according to claim 53, wherein the reaction mixture for RNA synthesis comprises at least one nucleoside triphosphate optionally labeled with a radioactive isotope or is chemically modified, pH buffer, ammonium acetate, PEG, Mg²⁺ ions, Mn²⁺ ions and/or non-ionic detergent.

60. The method according to claim 59, wherein the method is specifically used for producing radioactively or chemically labeled RNA probes and comprising an optional step of purifying the newly produced labeled RNA from the components of the reaction mixture.

61. The method according to claim 60, wherein said labeled RNAs are used as probes for Southern or Northern blot analyses after the optional purification step.

62. The method according to claim 60, wherein said labeled RNAs are used as probes for a fluorescent in situ hybridization analysis after the optional purification step.

63. The method according to claim 60, wherein said labeled RNAs are used as probes for a microarray analysis after the optional purification step.

64. An isolated polypeptide, characterized in that:
(i) said polypeptide has sufficient RNA polymerase activity and is capable, when contacted with a nucleic acid template, of producing short complementary RNA copies of said template, which RNA copies are scattered throughout the entire template length and, optionally, capable of producing template-length complementary RNA copies;
(ii) said polypeptide has enhanced solubility resulting in at least 3 times higher yield of the active polymerase, than in the case of polypeptide comprising the amino acid sequence of SEQ ID NO: 2 or encoded by the nucleic acid sequence comprising SEQ ID NO: 1; and
(iii) said polypeptide is encoded by a nucleic acid sequence selected from the group of:
(a) a nucleic acid sequence comprising the nucleic acid sequence of SEQ ID NO: 3;
(b) a nucleic acid sequence encoding a polypeptide comprising the amino acid sequence of SEQ ID NO: 4;
(c) a nucleic acid sequence, which differs from the nucleic acid sequence of (a) or (b) due to degeneracy of the genetic code;
(d) a nucleic acid sequence hybridizing to the nucleic acid sequence of (a), (b) and/or (c) under stringent conditions;
(e) a nucleic acid sequence encoding a polypeptide comprising the amino acids 709 to 1402 of SEQ ID NO:4 or any sequence longer than that up to the sequence comprising the amino acids 2 to 1402 of SEQ ID NO: 2; and
(f) a nucleic acid sequence encoding an amino acid sequence, which shows at least 50% identity to the amino acid sequence SEQ ID NO: 2.

65. An isolated nucleic acid sequence encoding the polypeptide according to claim 64.

66. A vector, which comprises the nucleic acid sequence of claim 65 operationally linked with regulatory sequences required for gene expression.

67. A host cell comprising the vector of claim 66.

68. A method for producing a polymerase protein, which comprises culturing the host cell of claim 67 under conditions suitable for the expression of the protein.

69. The method according to claim 68, comprising the step that the protein is recovered from the cell or culture medium and optionally purified.

70. A method for studying nucleic acid secondary structure, preferably RNA secondary structure comprising the steps of:
(a) providing nucleic acid target molecule, preferably RNA target molecule;
(b) contacting said target molecule with an isolated RNA polymerase capable of producing short complementary RNA copies of the nucleic acid target as template, which RNA copies are scattered throughout the ssRNA regions of the template length and, optionally, template-length complementary RNA copies under conditions sufficient for RNA synthesis in a mixture additionally comprising radioactively or chemically labeled nucleotides, so that single-stranded elements of said target RNA are copied by the polymerase;
(c) recovering and optionally purifying the newly produced labeled nucleic acid species from the reaction mixture;
(d) using said labeled nucleic acid species as probes for microarray chip that comprises nucleic acid fragments of said target molecule;
(e) interpreting data from the microarray analysis to deduce which parts of the target molecule are single-stranded; and optionally
(f) building a model for the secondary or tertiary structure of the target molecule.

71. A method for studying nucleic acid-protein interactions, preferably RNA-protein interactions comprising the steps of:
(a) providing a nucleic acid target and nucleic acid binding protein, preferably an RNA target and an RNA-binding protein;
(b) contacting said target and the solution of said protein in an experimental mixture under conditions sufficient for target-protein interaction, and in a separate vessel, contacting said target with a control solution that lacks said protein.
(c) contacting said experimental and control mixtures with an isolated RNA polymerase capable of producing short complementary RNA copies of said target as template, which RNA copies are scattered throughout the template length not covered by protein and, optionally, template-length complementary RNA copies under conditions sufficient for RNA synthesis;
(d) recovering and optionally purifying the newly produced labeled nucleic acid species from both reaction mixtures;
(e) using the two sets of labeled nucleic acid species as probes for two identical microarray chips that comprise nucleic acid fragments of the target;
(f) interpreting data from the two microarray analyses to deduce which parts of the target molecule are accessible for the RNA synthesis;
(g) comparing the two data sets to determine the difference between target in experimental and control mixtures; and optionally
(h) interpreting the difference between the two data sets as a model for nucleic acid-protein interactions

72. A method for producing RNA trigger molecules to induce RNA interference in vivo or in vitro, comprising the steps of:
(a) providing RNA or DNA template;
(b) contacting said RNA or DNA template with an isolated RNA polymerase capable of producing short complementary RNA copies of said template, which are scattered throughout the entire template length and, optionally, template-length complementary RNA copies under conditions sufficient for RNA synthesis in a mixture comprising: nucleic acid template, said RNA polymerase, nucleoside triphosphates, and optionally pH buffer, ammonium acetate, PEG, Mg²⁺ ions, Mn²+ ions and/or non-ionic detergent; and
(c) incubating the reaction mixture at temperature sufficient for RNA synthesis.

73. The method according to claim 72, wherein said RNA or DNA template originates from a cell or a virus.

74. The method according to claim 72, wherein said RNA template is provided by transcribing a DNA template with a DNA-dependent RNA polymerase, preferably derived from a bacteriophage selected from the group of T7, T3, and SP6 bacteriophages.

75. The method according to claim 72, wherein steps (a) and (b) are carried out at the same time or sequentially in the same reaction vessel.

76. A kit comprising the polypeptide of claim 64.

77. The kit according to claim 76, wherein the kit further comprises additives necessary for a detectable level of RNA synthesis.

78. The kit according to claim 76 comprising nucleoside triphosphates in concentrations sufficient for RNA synthesis.

79. The kit according to any claim 76, wherein at least one nucleoside triphosphate is labeled with a radioactive isotope or is chemically modified.

80. The kit according to claim 76, additionally comprising a standard nucleic acid preparation (or preparations) with characterized capacity to serve as a template (templates) for RNA synthesis by the polypeptide characterized in that:
(i) said polypeptide has sufficient RNA polymerase activity and is capable, when contacted with a nucleic acid template, of producing short complementary RNA copies of said template, which RNA copies are scattered throughout the entire template length and, optionally, capable of producing template-length complementary RNA copies;
(ii) said polypeptide has enhanced solubility resulting in at least 3 times higher yield of the active polymerase, than in the case of polypeptide comprising the amino acid sequence of SEQ ID NO: 2 or encoded by the nucleic acid sequence comprising SEQ ID NO: 1; and
(iii) said polypeptide is encoded by a nucleic acid sequence selected from the group of:
(a) a nucleic acid sequence comprising the nucleic acid sequence of SEQ ID NO: 3;
(b) a nucleic acid sequence encoding a polypeptide comprising the amino acid sequence of SEQ ID NO: 4;
(c) a nucleic acid sequence, which differs from the nucleic acid sequence of (a) or (b) due to degeneracy of the genetic code;
(d) a nucleic acid sequence hybridizing to the nucleic acid sequence of (a), (b) and/or (c) under stringent conditions;
(e) a nucleic acid sequence encoding a polypeptide comprising the amino acids 709 to 1402 of SEQ ID NO:4 or any sequence longer than that up to the sequence comprising the amino acids 2 to 1402 of SEQ ID NO: 2; and
(f) a nucleic acid sequence encoding an amino acid sequence, which shows at least 50% identity to the amino acid sequence SEQ ID NO: 2.

81. Use of the method of claim 42 for studying nucleic acid secondary structure, for studying nucleic acid-protein interactions, or for producing RNA trigger molecules.


SEQUENCE LISTING (15 sequences)

SEQ ID NO: 1 - DNA, 4206 bp, Neurospora crassa
atgaaccctattactcctaggaagaggaatagccccgtcgaggaaatcataaaccggctcaataacgactacaacctgggcctccagtgtgtcgcagacacaactctcaccccccaccgccggaaggagctggccgagagtgacgaggatttcggtcgccatgacaagatctacagagccctgaactttctctactggcggaaggatgactccctgaaccaggcagaagccaacttcttcatcgaggccaaagctgcgagctcgaactgggtgcccaaagcccacgccgaccctgacacgcttccgtggtccaaggaacctccccgcgccgctactgccggccaacaatgggcattgcagactgtgttgctcgaggtgcttaataggtttatgccacctcccaataacacaccaggtcgaacgtttggcagaactctaagcggcccaagtggcctgagccgcccaacctctaccaacaccaaacgcaaggatgagcccgccaatgtcactttcgctgatccgcccaaacgctcgttgactcgctctgccacaggtcctcctattcacggcgcggcgatacccctaaagttccccgatccagtgaataccggttccaaacgaccatctctcgagagtgagaatctcaatcagtgcaccaagcgggccaagggcaagctgtctgataatgttgccgctgccgccgccccgcccgtgcctattgcgagcgctttggacaaggtaccgactcgaaggcatgccaatacgagagatcccacggcgacaggtcatagacgagcggaccaggtggattcctttgatacatctcaaggcacttcctatggttcgagtgtcttcagcgcttgccgtcacaatcagagcactacccagagtagttttgaggcccctccttcacagcccagagagaagcggcctgtggatgccacggtctttgaggctggacacttgattgagtctcctagcaaaggaagaacaaccaagtcccacatagataaccagcccctttcatcgtcctcccagggtgaaacttcgttcagcacttactatgagtcgtttccaagttccggcggcgagggcgcaattcccgagccgagtcgctcaaatggactggctcggagcgaagaaagcgctcgatctcaggttcaagttcatgccccggtggttgcagctcggctgagaaatatttggccgaaatttcccaaatggctacacgaagctcctctcgctgttgcatgggaagttaccagactctttatgcactgcaaagtagacttggaagacgagagcctgggcctaaagtacgacccttcctggtctaccgcgcgcgatgtcacagatatctggaagactctctaccggcttgatgctttccgtggtaaaccctttccagaaaagccgcccaacgacgtgttcgtgaccgcaatgacgggcaactttgagagcaaaggtagtgccgttgttctctctgctgttctagactacaatccggacaactcgcctactgcgcccctttaccttgtgaagctgaagccgctcatgttcgagcagggctgtcgactcacccgtcggttcggtcctgataggtttttcgagatccttataccgtcgcctacgagcaccagcccaagtgtaccgccggtggtcagcaaacaaccaggtgcggtcgaagaagtcatccagtggctcacgatggggcaacattctcttgtaggccgccaatggcgcgctttcttcgccaaagatgccggatacaggaaacctctcagggagttccagctccgcgccgaggacccgaaacccatcatcaaggagagagtccacttctttgccgagaccggcattacgttccgacctgatgtgttcaagacgagatctgtcgttccggcagaggaacctgtagagcaacggaccgagttcaaagttagtcaaatgctggactggctcctgcaactcgacaacaacacttggcagccgcacctcaagttgttctcccgtatccagctcggtctgagtaagacatatgccattatgacattggagcctcaccagatcagacaccacaagaccgatcttctttcaccttcaggcactggcgaagtgatgaatgacggtgtaggccgcatgtcgcgaagcgtggccaagaggatacgcgatgttctcggtttgggtgatgtgccctctgctgtgcaagggcggtttggttcggccaagggaatgtgggttatcgacgttgacgacacaggcgatgaggattggatcgagacatacccgtcccagcgcaagtgggaatgcgacttcgttgataaacatcaacgtaccctcgaagtccggagcgtggcttctgaactgaagtcagctggtctcaacctacagctgttacctgtcctggaagatagagccagggacaaggtgaagatgcgccaggcaatcggtgaccgtcttatcaacgatttgcaacgacagttcagcgagcaaaagcatgctttgaatcgcccagtggaatttcgccaatgggtttacgagagttattccagtcgcgcaactcgagtcagccacggccgtgtgccttttcttgctgggctacctgacagtcaagaggagacactgaacttcttgatgaacagtgggttcgatcccaagaagcaaaagtacttgcaagacatcgcctgggatcttcaaaagcggaaatgtgacacgttgaagtccaagctgaacatccgtgtcggtcgatcagcatacatttacatgattgccgatttctggggtgtgcttgaggaaaatgaggttcatgtcggattctcctcaaagttcagagacgaggaggagtcttttacactcctatcggactgtgatgtcctcgtggcgcgatccccagcccatttccctagtgatatccaacgggttcgagcagtcttcaagccagagctccacagtctcaaggatgtaatcatcttctctactaaaggagatgtaccgcttgctaagaagctatctggtggagactacgacggcgatatggcctgggtctgctgggatccggagatcgtcgatggtttcgtcaatgcggaaatgcctctggagcccgacctgtctaggtacctaaagaaggacaaaacgactttcaaacaacttatggcctcacacggcacgggctcagcggccaaagagcagactacatacgatatgatccagaagagcttccatttcgccctgcagcccaacttcttgggcatgtgcactaactacaaagaaaggctctgttacatcaacaatagtgtgtctaacaagccggccatcattcttagttcactggtgggaaacctcgtcgatcagagcaagcaaggtattgtctttaacgaagcaagctgggctcaattgcgtagggaactgcttggcggtgcattgtcccttcctgacccaatgtacaagagcgacagttggctcgggcgcggagagcctacccacattattgactacctgaaattctccatcgccaggcctgcgattgacaaggaactggaagccttccacaatgccatgaaagcggccaaggatacagaagacggcgctcacttttgggatccggatctcgcttcctactacacgttcttcaaggagattagcgacaagtcgcgatcgtccgcactgctattcacgactctgaagaaccgtatcggcgaagtcgagaaagaatatggcaggttggtcaaaaacaaggagatgagagacagcaaggacccctaccctgtccgcgtcaaccaggtttatgaaaaatggtgcgccatcacgcctgaggcgatggacaaatccggagcaaattatgattctaaggtgatcaggttgctggagctgtccttcctcgcggaccgtgagatgaatacatgggcattgctgagggctagcacggctttcaagctgtactaccacaagagccccaagttcgtgtggcagatggcgggcagacagctcgcgtacattaaggcgcagatgacgagcagacccggtgaaggcgccccggcgttgatgaccgcgttcatgtatgcgggcttgatgccggataagaagtttacgaagcagtatgtggccaggctggagggcgatggatcggagtaccctgatccggaggtctatgaagtgctgggcgatgatgattttgatggaattggtttcacagggaatggcgattat

SEQ ID NO: 2 - protein, 1402 aa, Neurospora crassa
MNPITPRKRNSPVEEIINRLNNDYNLGLQCVADTTLTPHRRKELAESDEDFGRHDKIYRALNFLYWRKDDSLNQAEANFFIEAKAASSNWVPKAHADPDTLPWSKEPPRAATAGQQWALQTVLLEVLNRFMPPPNNTPGRTFGRTLSGPSGLSRPTSTNTKRKDEPANVTFADPPKRSLTRSATGPPIHGAAIPLKFPDPVNTGSKRPSLESENLNQCTKRAKGKLSDNVAAAAAPPVPIASALDKVPTRRHANTRDPTATGHRRADQVDSFDTSQGTSYGSSVFSACRHNQSTTQSSFEAPPSQPREKRPVDATVFEAGHLIESPSKGRTTKSHIDNQPLSSSSQGETSFSTYYESFPSSGGEGAIPEPSRSNGLARSEESARSQVQVHAPVVAARLRNIWPKFPKWLHEAPLAVAWEVTRLFMHCKVDLEDESLGLKYDPSWSTARDVTDIWKTLYRLDAFRGKPFPEKPPNDVFVTAMTGNFESKGSAVVLSAVLDYNPDNSPTAPLYLVKLKPLMFEQGCRLTRRFGPDRFFEILIPSPTSTSPSVPPVVSKQPGAVEEVIQWLTMGQHSLVGRQWRAFFAKDAGYRKPLREFQLRAEDPKPIIKERVHFFAETGITFRPDVFKTRSVVPAEEPVEQRTEFKVSQMLDWLLQLDNNTWQPHLKLFSRIQLGLSKTYAIMTLEPHQIRHHKTDLLSPSGTGEVMNDGVGRMSRSVAKRIRDVLGLGDVPSAVQGRFGSAKGMWVIDVDDTGDEDWIETYPSQRKWECDFVDKHQRTLEVRSVASELKSAGLNLQLLPVLEDRARDKVKMRQAIGDRLINDLQRQFSEQKHALNRPVEFRQWVYESYSSRATRVSHGRVPFLAGLPDSQEETLNFLMNSGFDPKKQKYLQDIAWDLQKRKCDTLKSKLNIRVGRSAYIYMIADFWGVLEENEVHVGFSSKFRDEEESFTLLSDCDVLVARSPAHFPSDIQRVRAVFKPELHSLKDVIIFSTKGDVPLAKKLSGGDYDGDMAWVCWDPEIVDGFVNAEMPLEPDLSRYLKKDKTTFKQLMASHGTGSAAKEQTTYDMIQKSFHFALQPNFLGMCTNYKERLCYINNSVSNKPAIILSSLVGNLVDQSKQGIVFNEASWAQLRRELLGGALSLPDPMYKSDSWLGRGEPTHIIDYLKFSIARPAIDKELEAFHNAMKAAKDTEDGAHFWDPDLASYYTFFKEISDKSRSSALLFTTLKNRIGEVEKEYGRLVKNKEMRDSKDPYPVRVNQVYEKWCAITPEAMDKSGANYDSKVIRLLELSFLADREMNTWALLRASTAFKLYYHKSPKFVWQMAGRQLAYIKAQMTSRPGEGAPALMTAFMYAGLMPDKKFTKQYVARLEGDGSEYPDPEVYEVLGDDDFDGIGFTGNGDY

SEQ ID NO: 3 - DNA, 3078 bp, Neurospora crassa
gctcggagcgaagaaagcgctcgatctcaggttcaagttcatgccccggtggttgcagctcggctgagaaatatttggccgaaatttcccaaatggctacacgaagctcctctcgctgttgcatgggaagttaccagactctttatgcactgcaaagtagacttggaagacgagagcctgggcctaaagtacgacccttcctggtctaccgcgcgcgatgtcacagatatctggaagactctctaccggcttgatgctttccgtggtaaaccctttccagaaaagccgcccaacgacgtgttcgtgaccgcaatgacgggcaactttgagagcaaaggtagtgccgttgttctctctgctgttctagactacaatccggacaactcgcctactgcgcccctttaccttgtgaagctgaagccgctcatgttcgagcagggctgtcgactcacccgtcggttcggtcctgataggtttttcgagatccttataccgtcgcctacgagcaccagcccaagtgtaccgccggtggtcagcaaacaaccaggtgcggtcgaagaagtcatccagtggctcacgatggggcaacattctcttgtaggccgccaatggcgcgctttcttcgccaaagatgccggatacaggaaacctctcagggagttccagctccgcgccgaggacccgaaacccatcatcaaggagagagtccacttctttgccgagaccggcattacgttccgacctgatgtgttcaagacgagatctgtcgttccggcagaggaacctgtagagcaacggaccgagttcaaagttagtcaaatgctggactggctcctgcaactcgacaacaacacttggcagccgcacctcaagttgttctcccgtatccagctcggtctgagtaagacatatgccattatgacattggagcctcaccagatcagacaccacaagaccgatcttctttcaccttcaggcactggcgaagtgatgaatgacggtgtaggccgcatgtcgcgaagcgtggccaagaggatacgcgatgttctcggtttgggtgatgtgccctctgctgtgcaagggcggtttggttcggccaagggaatgtgggttatcgacgttgacgacacaggcgatgaggattggatcgagacatacccgtcccagcgcaagtgggaatgcgacttcgttgataaacatcaacgtaccctcgaagtccggagcgtggcttctgaactgaagtcagctggtctcaacctacagctgttacctgtcctggaagatagagccagggacaaggtgaagatgcgccaggcaatcggtgaccgtcttatcaacgatttgcaacgacagttcagcgagcaaaagcatgctttgaatcgcccagtggaatttcgccaatgggtttacgagagttattccagtcgcgcaactcgagtcagccacggccgtgtgccttttcttgctgggctacctgacagtcaagaggagacactgaacttcttgatgaacagtgggttcgatcccaagaagcaaaagtacttgcaagacatcgcctgggatcttcaaaagcggaaatgtgacacgttgaagtccaagctgaacatccgtgtcggtcgatcagcatacatttacatgattgccgatttctggggtgtgcttgaggaaaatgaggttcatgtcggattctcctcaaagttcagagacgaggaggagtcttttacactcctatcggactgtgatgtcctcgtggcgcgatccccagcccatttccctagtgatatccaacgggttcgagcagtcttcaagccagagctccacagtctcaaggatgtaatcatcttctctactaaaggagatgtaccgcttgctaagaagctatctggtggagactacgacggcgatatggcctgggtctgctgggatccggagatcgtcgatggtttcgtcaatgcggaaatgcctctggagcccgacctgtctaggtacctaaagaaggacaaaacgactttcaaacaacttatggcctcacacggcacgggctcagcggccaaagagcagactacatacgatatgatccagaagagcttccatttcgccctgcagcccaacttcttgggcatgtgcactaactacaaagaaaggctctgttacatcaacaatagtgtgtctaacaagccggccatcattcttagttcactggtgggaaacctcgtcgatcagagcaagcaaggtattgtctttaacgaagcaagctgggctcaattgcgtagggaactgcttggcggtgcattgtcccttcctgacccaatgtacaagagcgacagttggctcgggcgcggagagcctacccacattattgactacctgaaattctccatcgccaggcctgcgattgacaaggaactggaagccttccacaatgccatgaaagcggccaaggatacagaagacggcgctcacttttgggatccggatctcgcttcctactacacgttcttcaaggagattagcgacaagtcgcgatcgtccgcactgctattcacgactctgaagaaccgtatcggcgaagtcgagaaagaatatggcaggttggtcaaaaacaaggagatgagagacagcaaggacccctaccctgtccgcgtcaaccaggtttatgaaaaatggtgcgccatcacgcctgaggcgatggacaaatccggagcaaattatgattctaaggtgatcaggttgctggagctgtccttcctcgcggaccgtgagatgaatacatgggcattgctgagggctagcacggctttcaagctgtactaccacaagagccccaagttcgtgtggcagatggcgggcagacagctcgcgtacattaaggcgcagatgacgagcagacccggtgaaggcgccccggcgttgatgaccgcgttcatgtatgcgggcttgatgccggataagaagtttacgaagcagtatgtggccaggctggagggcgatggatcggagtaccctgatccggaggtctatgaagtgctgggcgatgatgattttgatggaattggtttcacagggaatggcgattat

SEQ ID NO: 4 - protein, 1026 aa, Neurospora crassa
ARSEESARSQVQVHAPVVAARLRNIWPKFPKWLHEAPLAVAWEVTRLFMHCKVDLEDESLGLKYDPSWSTARDVTDIWKTLYRLDAFRGKPFPEKPPNDVFVTAMTGNFESKGSAVVLSAVLDYNPDNSPTAPLYLVKLKPLMFEQGCRLTRRFGPDRFFEILIPSPTSTSPSVPPVVSKQPGAVEEVIQWLTMGQHSLVGRQWRAFFAKDAGYRKPLREFQLRAEDPKPIIKERVHFFAETGITFRPDVFKTRSVVPAEEPVEQRTEFKVSQMLDWLLQLDNNTWQPHLKLFSRIQLGLSKTYAIMTLEPHQIRHHKTDLLSPSGTGEVMNDGVGRMSRSVAKRIRDVLGLGDVPSAVQGRFGSAKGMWVIDVDDTGDEDWIETYPSQRKWECDFVDKHQRTLEVRSVASELKSAGLNLQLLPVLEDRARDKVKMRQAIGDRLINDLQRQFSEQKHALNRPVEFRQWVYESYSSRATRVSHGRVPFLAGLPDSQEETLNFLMNSGFDPKKQKYLQDIAWDLQKRKCDTLKSKLNIRVGRSAYIYMIADFWGVLEENEVHVGFSSKFRDEEESFTLLSDCDVLVARSPAHFPSDIQRVRAVFKPELHSLKDVIIFSTKGDVPLAKKLSGGDYDGDMAWVCWDPEIVDGFVNAEMPLEPDLSRYLKKDKTTFKQLMASHGTGSAAKEQTTYDMIQKSFHFALQPNFLGMCTNYKERLCYINNSVSNKPAIILSSLVGNLVDQSKQGIVFNEASWAQLRRELLGGALSLPDPMYKSDSWLGRGEPTHIIDYLKFSIARPAIDKELEAFHNAMKAAKDTEDGAHFWDPDLASYYTFFKEISDKSRSSALLFTTLKNRIGEVEKEYGRLVKNKEMRDSKDPYPVRVNQVYEKWCAITPEAMDKSGANYDSKVIRLLELSFLADREMNTWALLRASTAFKLYYHKSPKFVWQMAGRQLAYIKAQMTSRPGEGAPALMTAFMYAGLMPDKKFTKQYVARLEGDGSEYPDPEVYEVLGDDDFDGIGFTGNGDY

SEQ ID NO: 5 - DNA, 30 bp, Artificial Sequence
  artificial sequence is primer
gtaatacgactcactataggccgtggtggt

SEQ ID NO: 6 - DNA, 30 bp, Artificial Sequence
  artificial sequence is primer
ttttctgaattctcttgccgcttttattct

SEQ ID NO: 7 - DNA, 30 bp, Artificial Sequence
  artificial sequence is primer
gccaagcttccatgaaccctattactccta

SEQ ID NO: 8 - DNA, 28 bp, Artificial Sequence
  artificial sequence is primer
ccgaattcataatcgccattccctgtga

SEQ ID NO: 9 - DNA, 30 bp, Artificial Sequence
  artificial sequence is primer
gctcaaatcccatggctcggagcgaagaaa

SEQ ID NO: 10 - DNA, 30 bp, Artificial Sequence
  artificial sequence is primer
ccgaattctaataatcgccattccctgtga

SEQ ID NO: 11 - RNA, 20 nt, Artificial Sequence
  artificial sequence is primer
guuuucacccuauccucccc

SEQ ID NO: 12 - RNA, 20 nt, Artificial Sequence
  artificial sequence is primer
cgacucauggaccuugggag

SEQ ID NO: 13 - protein, 8 aa, Neurospora crassa
  MISC_FEATURE (1)..(8): Xaa is any amino acid
GSDLDGDX

SEQ ID NO: 14 - DNA, 129 bp, Artificial Sequence
  artificial sequence is tag sequence
gaattctgcagatatccagcacagtggcggccgctcgagtctagagggcccttcgaaggtaagcctatccctaaccctctcctcggtctcgattctacgcgtaccggtcatcatcaccatcaccattga

SEQ ID NO: 15 - protein, 42 aa, Artificial Sequence
  artificial sequence is tag sequence
EFCRYPAQWRPLESRGPFEGKPIPNPLLGLDSTRTGHHHHHH